(19) United States
(12) Patent Application Publication
(10) Pub. No.: US 2025/0154237 A1
(43) Pub. Date: May 15, 2025

(54) FULLY HUMAN MONOCLONAL ANTIBODIES AGAINST HUMAN PROGRANULIN

(71) Applicant: A & G Pharmaceutical, Inc., Columbia, MD (US)

(72) Inventor: Ginette Serrero, Ellicott City, MD (US)

(21) Appl. No.: 18/838,875

(22) PCT Filed: Feb. 23, 2023
(86) PCT No.: PCT/US2023/063169

§ 371 Date: Aug. 15, 2024

Related U.S. Application Data
(60) Provisional application No. 63/313,304, filed on Feb. 24, 2022.

(51) Int. Cl. C07K 16/22; A61P 35/00; C12N 15/11; G01N 33/574; G01N 33/68

(52) U.S. Cl. CPC C07K 16/22; A61P 35/00; C12N 15/11; G01N 33/5748; G01N 33/68; C07K 2317/21; C07K 2317/51; C07K 2317/515; C07K 2317/565; C12N 2510/00

(57) ABSTRACT

Described herein are antibodies, particularly monoclonal antibodies, that specifically bind to human progranulin and are useful for the treatment of cancer in patients. Methods for preparing and using the same are also provided.

### RELATED APPLICATIONS

This application is a national stage application, filed under 35 U.S.C. § 371, of International Patent Application No. PCT/US2023/063169 filed on Feb. 23, 2023, which claims the benefit of U.S. Provisional Application Ser. No. 63/313,304, filed Feb. 24, 2022, the entire disclosure of which are incorporated herein by reference for all purposes.

### TECHNICAL FIELD

Herein provides fully human monoclonal antibodies (mAbs). More particularly, herein describes mAbs that specifically bind to human progranulin, also known as GP88. Other names for progranulin include granulin/epithelin precursor, GEP, and acrogranin.

### BACKGROUND INFORMATION

A promising novel therapeutic and diagnostic target for cancer was identified and called PC-Cell Derived Growth Factor (PCDGF) (also known as granulin-epithelin precursor [GEP or GP88] or progranulin). PCDGF (referred to herein as progranulin) is an 88 kDa glycoprotein containing a 20 kDa carbohydrate moiety and a 68 kDa core protein identical to the precursor for epithelin/granulin, a family of six double-cysteine-rich polypeptides that are derived by proteolytic processing of the progranulin precursor (Bhandari, V. et al., Proc Natl Acad Sci USA 89(5): 1715-1719, 1992). The first identified members of this family were 6 kDa polypeptide growth modulators, epithelins 1 and 2 isolated from rat kidney (Shoyab, M., et al., Proc Natl Acad of Sci 87(20): 7912, 1990) or granulins from human granulocyte extracts (Bateman et al. Biochem Biophys Res Commun 173(3): 1161-1168, 1990).

Cloning of the complementary deoxyribonucleic acid (cDNA) for the epithelins and granulins showed that it encoded a 63 kDa protein that contained 7.5 kDa cysteine-rich epithelin/granulin repeats, a signal peptide, and several putative glycosylation sites (Bhandari, V. et al., Proc Natl Acad Sci USA 89(5): 1715-1719, 1992). Studies on the biological function of the cloned and expressed precursor suggested that it was biologically inactive (Bhandari, V. et al., 1992 and Plowman, G. et al., J Biol Chem 267(18): 13073-13078, 1992). This led to the hypothesis that the precursor needed to be processed into the 6 kDa molecular forms in order to acquire biological activity. Based on this assumption, the large molecular weight form was defined as granulin/epithelin precursor or PGRN, now called progranulin. Under normal physiological conditions progranulin is not secreted but serves as the precursor to the mature granulins/epithelins. The work by the Serrero laboratory, reporting the purification of an autocrine growth factor from the culture medium of the highly tumorigenic teratoma PC cell line, provided the first demonstration of the physiological existence of the precursor as an 88 kDa glycoprotein with a 17 amino-acid signal peptide and a 20 kDa carbohydrate moiety as a biologically active growth factor (Zhou, G. et al., J Biol Chem 268(15): 10863-10869, 1993). In other studies of several cell lines of epithelial or mesenchymal origin (Zanocco-Marani, T., et al., Cancer Res 59(20): 5331-5340, 1999; He, Z. et al., Cancer Research 62(19): 5590, 2002; He, Z. et al., J Mol Med 81(10): 600-612, 2003; Serrero, G, Biochem Biophys Res Commun 308(3): 409-413, 2003) the acquisition of an autocrine production and secretion of progranulin resulted in deregulated growth leading to tumor formation in vivo indicating that progranulin acted as a tumorigenic growth factor.

Higher levels of autocrine progranulin expression are associated with breast cancer cells having the highest degree of malignancy. For instance, progranulin messenger ribonucleic acid (mRNA) and protein expression are very low in immortalized, non-tumorigenic, mammary epithelial MCF-10A cells and estrogen receptor positive (ER+) MCF-7 cells. In contrast, dramatically increased levels of progranulin are observed in more aggressive estrogen receptor negative (ER−) cell lines such as MDA-MB-468, 453 and 231 (Lu, R. et al., Proc Natl Acad Sci USA 97(8): 3993-3998, 2000). Further research has established that progranulin mediates the growth-promoting effect of 17-β estradiol (E2) in human breast cancer cells; in that, overexpression of progranulin in E2-dependent MCF-7 cells renders the cells E2-independent without change in E2 receptor status and E2 responsiveness, causing the cells to become tamoxifen-resistant (Lu, R. et al., Proc Natl Acad Sci USA 98(1): 142-147, 2001). Moreover, it has been discovered that tamoxifen-treated mice bearing progranulin overexpressing tumors form larger tumors than those in mice without tamoxifen treatment, indicating that ER+ breast cancer cells overexpressing progranulin not only became resistant to the growth inhibitory effect of tamoxifen, but they respond adversely to tamoxifen leading to the stimulation of tumor growth in vivo. In addition to conferring resistance to tamoxifen, data also demonstrate that overexpression of progranulin in ER+ breast cancer cells leads to resistance to fulvestrant (FASLODEX), aromatase inhibitor letrozole and chemotherapeutic agent doxorubicin (Tangkeangsirisin, W. et al., Carcinogenesis 25(9): 1587-1592, 2004; Abrhale; T. et al., BMC Cancer 11: 231, 2011; Tangkeangsirisin, W. et al., Advances in Breast Cancer Research Vol. 3 No. 3: 11, 2014). This latter finding is supported by the study by Kudoh et al (Kudoh, R. et al., Cancer Research 60(15): 4161, 2000) in which microarray analysis identified progranulin as a significantly (>12-fold) upregulated gene in doxorubicin-resistant breast cancer cells, MCF-7/D40. Also, progranulin was upregulated 17-fold in MCF-7 cells treated with doxorubicin for 15 hours. These results demonstrate that the increase in progranulin expression in breast tumors is associated with chemo-resistance.

Although little is known about the progranulin receptor as it has not been fully cloned, the progranulin-dependent signaling pathways that mediate proliferation and survival have been investigated by several laboratories (Arechavaleta-Velasco, F. et al., Med Oncol 34(12): 194, 2017). Concerning the receptor, specific, time- and temperature-dependent progranulin binding to cells that is saturable has been demonstrated. Cross-linking of biotinylated progranulin to human breast cancer cells using the cross-linker DSS showed the presence of a cross-linked band with an apparent molecular weight of about 190-200 kDa suggesting an apparent molecular weight of 100-110 kDa for the putative receptor (Xia et al., Biochem Biophys Res Commun 245(2): 539-543, 1998). Cells that do not bind or respond to progranulin did not present such a cross-linked protein complex. The intracellular signaling pathways mediated by progranulin include the MAPK ERK 1/2, PI-3 Kinase, and FAK, leading to the activation of the cell cycle regulatory proteins Cyclin D1 and Cyclin B (Zanocco-Marani, T. et al., Cancer Res 59(20): 5331-5340, 1999; Lu, R. et al., Proc Natl Acad Sci USA 98(1): 142-147, 2001; He, Z. et al., J Mol Med 81(10): 600-612, 2003; Jones, M. B. et al., Clin Cancer Res 9(1): 44-51, 2003). It has been shown that progranulin and its receptor can cross-talk with the Her-2 receptor and not the EGF receptor in Her-2 overexpressing breast cancer leading to phosphorylation of Her-2 and subsequently to Herceptin resistance (Kim, W. E. et al., Clin Cancer Res 12(14 Pt 1): 4192-4199, 2006).

Concerning the survival function of progranulin, examination of the mechanisms of action indicated that progranulin blocked the apoptotic effect of tamoxifen by upregulating bcl-2 expression and also strongly stimulated the expression of the angiogenic factor vascular endothelial growth factor (VEGF) and angiopoietin-2 (Tangkeangsirisin, W., et al., Carcinogenesis 25(9): 1587-1592, 2004). Since progranulin stimulated VEGF, an examination to determine whether progranulin also stimulated other processes that participate in metastasis was conducted. Progranulin, either overexpressed in MCF-7 cells or added exogenously, stimulated migration, expression of matrix metalloprotease-9 and invasion (Tangkeangsirisin, W. et al., Tangkeangsirisin, W. et al., Cancer Res 64(5): 1737-1743, 2004; Tangkeangsirisin, W. et al., Carcinogenesis 25(9): 1587-1592, 2004). Activation of MMP-2 in addition to MMP-9 has also been reported (He, Z. et al., J Mol Med 81(10): 600-612, 2003). Thus, progranulin stimulates angiogenesis and tumor cell invasiveness mediated, at least in part, by stimulating VEGF and matrix metalloproteinase expression, respectively (He, Z. et al., Cancer Research 62(19): 5590, 2002; He et al., 2003; Tangkeangsirisin, W. et al., Cancer Res 64(5): 1737-1743, 2004; Tangkeangsirisin, W. et al., Carcinogenesis 25(9): 1587-1592, 2004). These data suggest that an increased progranulin level is associated with increased metastasis.

Recently, a human/mouse chimeric monoclonal antibody named AG01, which specifically binds to human progranulin, was shown to have in vivo efficacy. AG01 treatment of mice bearing MDA-MB-231 subcutaneous tumors showed a significant reduction in tumor growth rate and tumor weight while body and organ weights were unaffected (Guha, R. et al., Breast Cancer Res Treat 186(3): 637-653, 2021).

Therefore, there exists a need for mAbs against human progranulin, particularly fully human mAbs that specifically bind human progranulin and which are useful in treating human cancers in patients.

### SUMMARY OF THE DISCLOSURE

Provided herein are monoclonal antibodies, or antigen binding fragments which bind progranulin (PGRN, or GP88). The isolated antibodies, or antigen binding fragments, described herein, bind human progranulin (GP88) with a K_{D} of less than or equal to 100 pM. For example, the isolated antibodies or antigen binding fragments described herein may bind to human progranulin with a K_{D} of less than or equal to 10 pM, less than or equal to 1 pM, less than or equal to 0.1 pM, or less than or equal to 0.01 pM. More specifically, the isolated antibodies or antigen binding fragments described herein may also bind human progranulin with a K_{D} of less than or equal to 0.05 pM or less than or equal to 0.4 pM, as measured by Octet assay described below or as measured by any assay available to the skilled artisan. Certain mAbs herein do not compete with mAb AG01, a chimeric monoclonal antibody that contains (a) mouse variable regions that specifically binds to human progranulin and (b) a human constant region (Guha, R. et al., 2021). Described herein is an isolated antibody, or antigen binding fragment thereof, that binds human progranulin and further competes for binding with mAbs 16C11, 10B3, 10C8 or 14A6 as described herein. Described herein is an isolated antibody, or antigen binding fragment thereof, that binds the same epitope as mAbs 16C11, 10B3, 10C8 or 14A6. In particular, the mAbs herein include the anti-human progranulin antibodies having the amino acid sequences (presented using one letter abbreviations standard in the field) recited in Table 1. These variable regions can be linked to IgG1/kappa isotype constant regions. Chothia and Kabat numbering systems for CDR residues are well known in the art and are further described in Dondelinger, M., et al., Front Immunol 9:2278, 2018, and were used to determine the amino acid sequences presented in Table 1 below. Exemplary cDNA sequences encoding the variable regions (VH and VL) are also presented below. The monoclonal antibodies can further include the complete heavy and/or light chain of the antibodies described in Table 1. In some embodiments, the monoclonal antibodies can include antigen-binding domains of such heavy and/or light chains, such as the CDRs shown in Table 1. In some embodiments, this disclosure provides nucleotide sequences encoding a 10C8, 16C11, 14A6, or 10B3 antibody (or functional fragment thereof such as a CDR and/or variable region thereof), and/or a particular amino acid sequence of an equivalent to an, wherein equivalents may be easily derived from the amino acid sequence of any of SEQ ID NOS. 1-56 and the information presented in Table 4, as well as using the functional and other assays disclosed herein, and/or as may be otherwise available to those of ordinary skill in the art. In some embodiments, this disclosure also provides expression vectors including an isolated nucleic acid comprising and/or consisting of such nucleotide sequences (in preferred embodiments any of SEQ ID NOS. 57-64 or derivatives thereof) as well as host cells (e.g., a cell line) containing such an expression vector. What is described herein relates to an isolated nucleic acid encoding a heavy or light chain of the monoclonal antibodies herein, an expression vector including the isolated nucleic acid and a cell line containing an expression vector. Also described herein are methods of treating cancer in a patient by identifying a patient having cancerous cells expressing progranulin and administering to the patient an antibody or antigen binding fragment as described herein. A variety of human cancers are known to express progranulin, including ovarian, breast, multiple myelomas, lung, renal carcinoma, prostate, hepatocellular carcinoma, uterine, bladder, biliary, esophageal, gastric, laryngeal, brain, leukemia and glioblastoma. Unless defined otherwise, all technical and scientific terms used herein have the same meaning as commonly understood by those of ordinary skill in the art. It is to be understood that both the foregoing general description and the following detailed description are exemplary and explanatory only and are not restrictive. Additional features will be set forth in part in the description which follows or may be learned by practice as described herein. The foregoing and other features will become apparent to one skilled in the art upon consideration of the following description of exemplary embodiments.

### BRIEF DESCRIPTION OF THE DRAWINGS

The accompanying drawings and photographs, which are incorporated in and constitute a part of this specification, illustrate several embodiments and together with the description serve to explain the principles herein. It is noted that AG01 is the same mAb as c4F10 recited herein, including in the Figures.

FIG. 1 is a bar graph showing flow binding to MDA-MB-231 cells for different anti-progranulin (PRGN or GP88) mAbs at different mAb concentrations.

FIG. 2 Is a bar graph showing percentage migration of MDA-MB-231 cells for different anti-progranulin mAbs.

FIG. 3 is a bar graph showing tumor volume after treatment with specified antibodies in an in vivo tumor growth model. The model uses MDA-MB-231 cells injected into athymic mice. The term “ip” refers to intraperitoneal injection of the therapeutic mAbs herein.

FIG. 4 is a line graph showing tumor volume when mice are treated using with new fully human mAb 10C8.

### DETAILED DESCRIPTION

This disclosure relates to antibodies, including but not limited to fully human monoclonal antibodies, that specifically bind to human progranulin (hu PRGN, or PRGN, or GP88) and which can be used to treat cancer, such as but not limited to breast cancer (e.g., triple negative breast cancer (can be exemplified using, e.g., the MDA-MB-231 cell line), adenocarcinoma epidermal growth factor receptor (EGF-R) breast cancer (can be exemplified using, e.g., the MDA-MB-468 cell line), basal breast carcinoma (can be exemplified using, e.g., the HS578T cell line), estrogen receptor (ER) luminal breast cancer (can be exemplified using, e.g., the MCF-7 cell line), ER positive tamoxifen resistant luminal breast cancer (can be exemplified using, e.g., the TamR MCF-7 cell line), letrozole resistant luminal breast cancer (can be exemplified using, e.g., the ACLRTUSM cell line)), ovarian cancer, uterine cancer, prostate cancer, bladder cancer (can be exemplified using, e.g., the T24 cell line), kidney cancer, hepatocellular cancer, biliary cancer, esophogeal cancer, colorectal cancer, gastric cancer, laryngeal cancer, lung cancer (e.g., non-small-cell lung carcinoma (can be exemplified using, e.g., the H1299 cell line), lung adenocarcinoma (can be exemplified using, e.g., the A549 cell line), brain cancer, myeloma/leukemia, mesothelioma (can be exemplified using, e.g., the MSTO-H11 cell line), epidermoid or squamous carcinoma (can be exemplified using, e.g., the A431 cell line), among others recognized to or found by those of skill in the art to express progranulin (GP88). In preferred embodiments, an exemplary cell line (e.g., one that can exemplify a cancer) is one in which, in a standard assay, is known and can be shown to express and/or bind progranulin (GP88), to be bound by or have progranulin (GP88) binding inhibited by an antibody and/or derivative of this disclosure, and/or to respond to the antibody and/or derivative in vitro (e.g., using migration assay) and/or in vivo (e.g., using a xenograft). In some embodiments, this disclosure provides an isolated antibody, one or more antigen binding fragment(s) thereof, that binds human progranulin (GP88) and further competes for binding with antibodies 10C8, 16C11, 14A6, or 10B3 antibody (or functional fragment thereof such as a CDR and/or variable region thereof); the heavy and light chain variable regions thereof (see Table 1); and/or a polypeptide comprising the CDRs of such antibodies (see Table 1); and/or derivatives thereof (e.g., comprising conservative amino acid substitutions thereto (see, e.g., Table 4)), any of which would be considered an equivalent thereof. In particular, the mAbs herein include the anti-human progranulin (GP88) antibodies having the amino acid sequences for the respective CDRs recited in Table 1, or variants thereof as disclosed herein or would be otherwise recognized by those of ordinary skill in the art.

In preferred embodiments, the antibodies of this disclosure comprise the following amino acid and nucleotide sequences. The preferred antibodies of this disclosure may comprise complementarity determining regions (CDRs) of the 10C8, 16C11, 14A6, or 10B3 antibodies. Table 1 presents the CDRs of the 10C8, 16C11, 14A6, or 10B3 antibodies using the Chothia and Kabat methods.

TABLE 1
Monoclonal Antibodies CDRs

| Antibody | 10C8 | 16C11 | 14A6 | 10B3 |
|---|---|---|---|---|
| H-CDR1 | GYTLTSY (SEQ | GFTFSSY | GFTFSNY | GFTLSGY |
| (Chothia) | ID NO: 1) | (SEQ ID | (SEQ ID | (SEQ ID NO: 43) |
| | | NO: 15) | NO: 29) | |
| H-CDR2 | SAYNGN (SEQ | WYDGSK | WYDGSN | WYDGSD (SEQ |
| (Chothia) | ID NO: 2) | (SEQ ID | (SEQ ID | ID NO: 44) |
| | | NO: 16) | NO: 30) | |
| H-CDR3 | ELLWFGELLS | DRGIGGDY | GHSVSGGDY | DRGAVAGDY |
| (Chothia) | QYYYYGMDV | (SEQ ID | (SEQ ID | (SEQ ID NO: 45) |
| | (SEQ ID NO: 3) | NO: 17) | NO: 31) | |
| L-CDR1 | RASQNISRWL | KSSQTVLY | RASQSVNSF | KSSQSVLYNS |
| (Chothia) | A (SEQ ID | SSNNKNYL | LN (SEQ ID | NNKNLLA |
| | NO: 4) | A (SEQ ID | NO: 32) | (SEQ ID NO: 46) |
| | | NO: 18) | | |
| L-CDR2 | RASSLET (SEQ | WASTRES | AASSLQS | WASTRES |
| (Chothia) | ID NO: 5) | (SEQ ID | (SEQ ID | (SEQ ID NO: 47) |
| | | NO: 19) | NO: 33) | |
| L-CDR3 | QQYNSYYT | QQYYSTPY | QQSYSTPYT | QQYYGTPYT |
| (Chothia) | (SEQ ID NO: 6) | T (SEQ ID | (SEQ ID | (SEQ ID NO: 48) |
| | | NO: 20) | NO: 34) | |
| H-CDR1 | SYGIS (SEQ ID | SYAMH | NYGMH (SEQ | GYGMH (SEQ |
| (Kabat) | NO: 7) | (SEQ ID | ID NO: 35) | ID NO: 49) |
| | | NO: 21) | | |
| H-CDR2 | WISAYNGNTN | VIWYDGSK | VIWYDGSNK | VIWYDGSDKN |
| (Kabat) | YAQKLQG | KYNADSVK | YYADSVKG | YADSVKG |
| | (SEQ ID NO: 8) | G (SEQ ID | (SEQ ID | (SEQ ID NO: 50) |
| | | NO: 22) | NO: 36) | |
| H-CDR3 | ELLWFGELLS | DRGIGGDY | GHSVSGGDY | DRGAVAGDY |
| (Kabat) | QYYYYGMDV | (SEQ ID | (SEQ ID | (SEQ ID NO: 51) |
| | (SEQ ID NO: 9) | NO: 23) | NO: 37) | |
| L-CDR1 | RASQNISRWL | KSSQTVLY | RASQSVNSF | KSSQSVLYNS |
| (Kabat) | A (SEQ ID | SSNNKNYL | LN (SEQ ID | NNKNLLA |
| | NO: 10) | A (SEQ ID | NO: 38) | (SEQ ID NO: 52) |
| | | NO: 24) | | |
| L-CDR2 | RASSLET (SEQ | WASTRES | AASSLQS | WASTRES |
| (Kabat) | ID NO: 11) | (SEQ ID | (SEQ ID | (SEQ ID NO: 53) |
| | | NO: 25) | NO: 39) | |
| L-CDR3 | QQYNSYYT | QQYYSTPY | QQSYSTPYT | QQYYGTPYT |
| (Kabat) | (SEQ ID NO: 12) | T (SEQ ID | (SEQ ID | (SEQ ID NO: 54) |
| | | NO: 26) | NO: 40) | |
| VH | QVQLVQSGAE | QVQLVESG | QVQLVESGG | QVQLVESGGG |
| | VKKPGASVKV | GGVVQPGR | GVVQPGRSL | WVQPGRSLRL |
| | SCKASGYTLTS | SLRLSCAA | RLSCAASGF | SCAASGFTLS |
| | YGISWVRQAP | SGFTFSSY | TFSNYGMHW | GYGMHWVRQ |
| | GQGLEWMGW | AMHWVRQ | VRQAPGKGL | APDKGLEWVA |
| | ISAYNGNTNYA | APGKGLEW | EWVAVIWYD | VIWYDGSDKN |
| | QKLQGRVTMT | VAVIWYDG | GSNKYYADS | YADSVKGRFTI |
| | TDTSTSTAYM | SKKYNADS | VKGRFTISRD | SRDNSKNTLYL |
| | ELRSLRSDDTA | VKGRFTISR | NSKNTLYLQ | QMNSLRAEDT |
| | VYYCARELLW | DNSKNTLY | MNSLRAEDT | AVYYCARDRG |
| | FGELLSQYYYY | LQMNSLRA | AVYYCARGH | AVAGDYWGQ |
| | GMDVWGQGT | EDTAVYYC | SVSGGDYW | GTLVTVSS |
| | TVTVSS (SEQ | ARDRGIGG | GQGTLVTVS | (SEQ ID NO: 55) |
| | ID NO: 13) | DYWGQGTL | S (SEQ ID | |
| | | VTVSS | NO: 41) | |
| | | (SEQ ID | | |
| | | NO: 27) | | |
| VL | DIQMTQSPSTL | DIVMTQSP | DIQMTQSPS | DIVMTQSPDSL |
| | SASVGDRVTIT | DSLAVSLG | SLSASVGDR | AVSLGERATIN |
| | CRASQNISRW | ERATINCKS | VTITCRASQS | CKSSQSVLYN |
| | LAWYQQKPGK | SQTVLYSS | VNSFLNWYQ | SNNKNLLAWF |
| | APKLLIFRASSL | NNKNYLAW | QTPGKAPKL | RQKPGQPPKL |
| | ETGVPSRFGG | YQQKPGQP | LIYAASSLQS | LIYWASTRESG |
| | SGSGTEFTLTI | PKLLIYWAS | GVPPRFSGS | VPDRFSGSGS |
| | SSLQPDDFAT | TRESGVPD | GSGTDFTLTI | GTDFTLTISSL |
| | YYCQQYNSYY | RFSGGGSG | SSLQPEDFA | QAEDVAVYFC |
| | TFGQGTRLEIK | TDFTLTISS | TYYCQQSYS | QQYYGTPYTF |
| | (SEQ ID NO: 14) | LQAEDVAV | TPYTFGQGT | GQGTKLEIK |
| | | YYCQQYYS | KLEIN (SEQ | (SEQ ID NO: 56) |
| | | TPYTFGQG | ID NO: 42) | |
| | | TKLEIN | | |
| | | (SEQ ID | | |
| | | NO: 28) | | |

In some embodiments, this disclosure provides fully human anti-progranulin antibodies that are internalized into cells expressing progranulin and compete with trastuzumab for binding to HER2, as well as methods for using such antibodies for their neutralizing as well as internalizing properties. In preferred embodiments, such antibodies include those referred to herein as 10C8, 16C11, 14A6, or 101B3, and/or the CDRs comprising by such antibodies (see Table 1); the heavy and light chain variable regions thereof (see Table 1); and/or derivatives thereof (e.g., comprising conservative amino acid substitutions thereto (see, e.g., Table 4)). In some embodiments, this disclosure provides fully human anti-progranulin antibodies that are internalized and do not compete with the antibodies or derivatives disclosed herein for binding to the progranulin, as well as methods for studying their internalizing properties. In preferred embodiments, such antibodies include those referred to herein as 10C8, 16C11, 14A6, or 10B3; comprising the heavy and light chain variable regions thereof (see Table 1); and/or comprising the CDRs of Table 1; and/or derivatives thereof (e.g., comprising conservative amino acid substitutions thereto (see, e.g., Table 4)). The antibodies can further include at least a portion of (most preferably including the CDRs thereof) and/or the complete heavy and/or light chain of the antibodies shown in Table 1, and/or a derivative thereof, that include the CDRs shown in Table 1. In some preferred embodiments, such antibodies can have the amino acid sequences of the variable heavy (“VH”) or variable light (“VL”) polypeptides (VH or VL “chains”, respectively) shown below for the antibody or equivalent thereof shown in Table 1. In some embodiments, the isolated antibodies, or antigen binding fragments, described herein, bind human progranulin with a K_{D} of up to 3.6×10-⁹ molar (M)), as measured by Octet assay described below (see, e.g., the Examples section herein), or as measured by any assay available to the skilled artisan. In some embodiments, this disclosure provides nucleotide sequences encoding a particular amino acid sequence of an equivalent to an 10C8, 16C11, 14A6, or 10B3 antibody may be easily derived from the amino acid sequence of any of Table 1 (i.e., SEQ ID NOS. 1-56) and the information presented in Table 4, and/or equivalents/derivatives thereof. In preferred embodiments, a 10C8 antibody comprises SEQ ID NOS. 1-3 and 7-9; or SEQ ID NOS. 4-6 and 10-12; and/or SEQ ID NO. 13 and SEQ ID NO. 14; or variants thereof as disclosed herein or as would be otherwise recognized by those of ordinary skill in the art. In preferred embodiments, a 16C11 antibody comprises SEQ ID NOS. 15-17 and 21-23; or SEQ ID NOS. 18-20 and 24-26; and/or SEQ ID NO. 27 and SEQ ID NO. 28; or variants thereof as disclosed herein or as would be otherwise recognized by those of ordinary skill in the art. In preferred embodiments, a 14A6 antibody comprises SEQ ID NOS. 29-31 and 35-37; or SEQ ID NOS. 32-34 and 38-40; and/or SEQ ID NO. 41 and SEQ ID NO. 42; or variants thereof as disclosed herein or as would be otherwise recognized by those of ordinary skill in the art. In preferred embodiments, a 10B3 antibody comprises SEQ ID NOS. 43-45 and 49-51; or SEQ ID NOS. 46-48 and 52-54; and/or SEQ ID NO. 55 and SEQ ID NO. 56; or variants thereof as disclosed herein or as would be otherwise recognized by those of ordinary skill in the art.

In some embodiments, this disclosure also provides expression vectors including an isolated nucleic acid comprising and/or consisting of such nucleotide sequences (in preferred embodiments as shown below, or derivatives thereof) as well as host cells (e.g., a cell line) containing such an expression vector. In some embodiments, this disclosure provides methods for using the antibodies and/or fragments thereof (e.g., CDRs) (which are referred to collectively as “antibodies” herein unless otherwise indicated) for treating cancers involving progranulin. In some embodiments, this disclosure also provides methods of treating cancer in a patient by identifying a patient having cancerous cells expressing progranulin and administering to the patient an antibody or antigen binding fragment as described herein. A variety of human cancers are known to express progranulin, including but not limited to ovarian, breast, multiple myelomas, lung, renal carcinoma, prostate, hepatocellular carcinoma, uterine, bladder, biliary, esophageal, gastric, laryngeal, brain, leukemia and glioblastoma. Thus, provided herein, in some preferred embodiments, are fully human monoclonal antibodies that specifically bind to human progranulin (PGRN) and which can be used to treat cancer, such as breast cancer.

The use of combinations of antibodies, such as one or more described herein with another available to those of ordinary skill in the art, are also contemplated herein. For instance, in some embodiments, the combinations may be identified to provide statistically significant differences from results (e.g., neutralization assays) obtained using only one or more of the antibodies and not others. In some embodiments, combinations exhibit additive and/or, preferably synergistic, activity. In some embodiments, the combination may comprise a 10C8, 16C11, 14A6, or 10B3 antibody (or derivative thereof) and other antibodies and/or conjugates. Combination can also be with chemotherapeutic agents used in the standard of care some of which are used in combination with anti-progranulin therapies. The antibodies of such compositions may be different entities such as two or more different monoclonal antibodies or derivatives thereof or may be found on the same entity such as a bi-functional antibody (a single antibody or derivative thereof comprising multiple binding specificities). Such combinations as described herein may also be combined with one or more other agents that may affect immune cell function such as antibodies against CTLA-4, and the like. One of ordinary skill in the art would recognize that many such combinations may be suitable for use as described herein.

The term “antibody” as used herein means a whole antibody and any antigen binding fragment (i.e., “antigen-binding portion”) or single chain thereof. A whole antibody is a glycoprotein comprising at least two heavy (H) chains and two light (L) chains inter-connected by di-sulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors. Isotype also refers to the antibody class (e.g., kappa, lambda) that is provided by the light-chain constant regions. The antibody may contain an Fc region including one or more mutations that influence one or more antibody properties, such as stability, pattern of glycosylation or other modifications, effector cell function, pharmacokinetics, and so forth. In some embodiments, an antibody has reduced or minimal glycosylation. In some embodiments, an antibody has ablated or reduced effector function. Exemplary Fc mutations include without limitation (i) a human IgG1 Fc region mutations L234A, L235A, G237A, and N297A; (ii) a human IgG2 Fc region mutations A330S, P331S and N297A; and (iii) a human IgG4 Fc region mutations S228P, E233P, F234V, L235A, delG236, and N297A (EU numbering). In some embodiments, the human IgG2 Fc region comprises A330S and P331S mutations. In some embodiments, the human IgG4 Fc region comprises an S288P mutation. In some embodiments, the human IgG4 Fc region comprises S288P and L235E mutations. Antibodies that target cell surface antigens can trigger immunostimulatory and effector functions that are associated with Fc receptor (FcR) engagement on immune cells. There are a number of Fc receptors that are specific for particular classes of antibodies, including IgG (gamma receptors), IgE (eta receptors), IgA (alpha receptors) and IgM (mu receptors). Binding of the Fc region to Fc receptors on cell surfaces can trigger a number of biological responses including phagocytosis of antibody-coated particles (antibody-dependent cell-mediated phagocytosis, or ADCP), clearance of immune complexes, lysis of antibody-coated cells by killer cells (antibody-dependent cell-mediated cytotoxicity, or ADCC) and release of inflammatory mediators, placental transfer, and control of immunoglobulin production. Additionally, binding of the C1 component of complement to antibodies can activate the complement system. Activation of complement can be important for the lysis of cellular pathogens. However, the activation of complement can also stimulate the inflammatory response and can also be involved in autoimmune hypersensitivity or other immunological disorders. Variant Fc regions with reduced or ablated ability to bind certain Fc receptors are useful for developing therapeutic antibodies and Fc-fusion polypeptide constructs which act by targeting, activating, or neutralizing ligand functions while not damaging or destroying local cells or tissues. An Fc domain monomer refers to a polypeptide chain that includes second and third antibody constant domains (e.g., CH2 and CH3). In some embodiments, an Fc domain monomer also includes a hinge domain. In some embodiments, the Fc domain monomer is of any immunoglobulin antibody isotype, including IgG, IgE, IgM, IgA, and IgD. Additionally, in some embodiments, an Fc domain monomer is of any IgG subtype (e.g., IgG1, IgG2, IgG2a, IgG2b, IgG2c, IgG3, and IgG4). Additional mutations in the Fc domain and the biological consequences of those mutations are well known in the art and can be applied to the antibodies herein. See, e.g., US Patent Application Publication No. 20220002434.

The term “antigen binding portion” or “antigen binding fragment” of an antibody, as used herein, refers to one or more fragments of an intact antibody that retain the ability to specifically bind to a given antigen (e.g., human granulin). Antigen binding functions of an antibody can be performed by fragments of an intact antibody. Examples of binding fragments encompassed within the term antigen binding portion or antigen binding fragment of an antibody include a Fab fragment, a monovalent fragment consisting of the V_{L}, V_{H}, CL and CH1 domains; a F(ab)₂ fragment, a bivalent fragment comprising two Fab fragments linked by a disulfide bridge at the hinge region; an Fd fragment consisting of the V_{H} and CH1 domains; an Fv fragment consisting of the V_{L} and V_{H} domains of a single arm of an antibody; a single domain antibody (dAb) fragment (Ward et al., 1989 Nature 341:544-546), which consists of a V_{H} domain or a V_{L} domain; and an isolated complementarity determining region (CDR). Furthermore, although the two domains of the Fv fragment, V_{L} and V_{H}, are coded for by separate genes, they can be joined, using recombinant methods, by an artificial peptide linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more antigen binding portions or fragments of an antibody. These antibody fragments are obtained using conventional techniques known to those of skill in the art, and the fragments are screened for utility in the same manner as are intact antibodies. Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies). Antigen binding fragments can be incorporated into single chain molecules comprising a pair of tandem Fv segments (V_{H}-CH1-V_{H}-CH1) which, together with complementary light chain polypeptides, form a pair of antigen binding regions (Zapata et al., 1995 Protein Eng. 8(10):1057-1062; and U.S. Pat. No. 5,641,870).

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. The term “binding specificity” as used herein refers to the ability of an individual antibody combining site to react with (e.g., have affinity for) only one antigenic determinant (e.g., epitope(s)). The phrase “specifically (or selectively) binds” to an antibody (e.g., an human progranulin-binding antibody) refers to a binding reaction that is determinative of the presence of a cognate antigen in a heterogeneous population of proteins and other biologics. The phrases “an antibody recognizing an antigen” and “an antibody specific for an antigen” are used interchangeably herein with the term “an antibody which binds specifically to an antigen”. As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. The term “K_{assoc}” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “K_{D}”, as used herein, is intended to refer to the dissociation constant, which is obtained from the ratio of Kd to Ka (i.e., Kd/Ka) and is expressed as a molar concentration (M). K_{D} values for antibodies can be determined using methods well established in the art. Methods for determining the K_{D} of an antibody include measuring surface plasmon resonance using a biosensor system such as a Biacore system, or measuring affinity in solution by solution equilibrium titration (SET). As used herein, the term “high affinity” for an antibody or antigen binding fragment thereof (e.g., a Fab fragment) generally refers to an antibody, or antigen binding fragment, having a K_{D} of 10⁻⁹ M or less.

The term “isolated antibody” refers to an antibody that is substantially free of other antibodies having different antigenic specificities (e.g., an isolated antibody that specifically binds progranulin is substantially free of antibodies that specifically bind antigens other than progranulin). An isolated antibody that specifically binds progranulin may, however, have cross-reactivity to other antigens, e.g., progranulin from species other than human. Moreover, an isolated antibody may be substantially free of other cellular material and/or chemicals. The terms “monoclonal antibody” or “monoclonal antibody composition” as used herein refer to a preparation of antibody molecules of single molecular composition. A monoclonal antibody composition displays a single binding specificity and affinity for a particular epitope. The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies may include amino acid residues not encoded by human sequences (e.g., mutations introduced by random or site-specific mutagenesis in vitro or by somatic mutation in vivo). The term “human monoclonal antibody” refers to antibodies displaying a single binding specificity which have variable regions in which both the framework and CDR regions are derived from human sequences. In one embodiment, the human monoclonal antibodies are produced by hybridomas which include (i) a B cell obtained from a transgenic non-human animal, e.g., a transgenic mouse, having a genome comprising a human heavy chain transgene and a light chain transgene (ii) fused to an immortalized cell. A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun., 28:489-498, 1991; and Padlan, Molec. Immun., 31:169-217, 1994. Other examples of human engineering technology include, but are not limited to, Xoma technology disclosed in U.S. Pat. No. 5,766,886.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

Amino acid substitutions considered conservative and non-conservative using standard three letter or other abbreviations for amino acids as would be understood by those of skill in the art are shown below in Table 2:

TABLE 2

| Original | Exemplary Conservative | Preferred Conservative |
|---|---|---|
| Amino | Substitutions of | Substitution of |
| Acid | the Original | the Original |
| Residue | Amino Acid Residue | Amino Acid Residue |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, Val, | Ile |
| | Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

The terms “identical” or 100% percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or over a region that is 100 to 500 or 1000 or more nucleotides (or 20, 50, 200 or more amino acids) in length. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001. The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available on the world wide web at gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

It is preferred that the antibody, or the antigen binding fragment thereof, comprises one or more amino acid sequences having at least 70%, at least 75%, at least 80%, at least 85%, at least 88%, at least 90%, at least 92%, at least 95%, at least 96%, at least 97%, at least 98% or at least 99% identity to at least one of SEQ ID NOs. 1-56 (i.e., the CDR sequences, the VH sequence and/or the VL sequences shown in Table 1). In some embodiments, an equivalent to an 10C8, 16C11, 14A6, or 10B3 antibody includes a derivative of one or more of the CDRs of the 10C8, 16C11, 14A6, or 10B3 antibodies, preferably including up to three (3) conservative amino acid substitutions of the CDRs thereof (see Table 2), provided the derivatives maintain their ability to bind to PGRN (preferably human PGRN (GP88)). In some embodiments, an equivalent to an 10C8, 16C11, 14A6, or 10B3 antibody includes a derivative of one or more of the VH and/or VL chains of the 10C8, 16C11, 14A6, or 10B3 antibodies, preferably including up to ten conservative amino acid substitutions outside of the CDRs thereof (see Table 2), provided the derivatives maintain the ability to bind to PGRN (preferably huPGRN). In preferred embodiments, any such substitutions allow for conjugation of the antibodies, or do not interfere with conjugation of the antibodies, to one or more detectable label(s), cytotoxic agent(s), and/or other payload (e.g., to provide a bi-specific antibody).

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

In certain embodiments, a nucleic acid molecule encoding one or more antibodies described herein may be inserted into one or more expression vectors, as discussed below in greater detail. In such embodiments, the antibody may be encoded by nucleotides corresponding to the amino acid sequence. The particular combinations of nucleotides (codons) that encode the various amino acids (AA) are well known in the art, as described in various references used by those skilled in the art (e.g., Lewin, B. Genes V, Oxford University Press, 1994). The nucleotide sequences encoding the amino acids of said antibodies may be ascertained with reference to Table 3, for example. Nucleic acid variants may use any combination of nucleotides that encode the antibody.

TABLE 3
Codons Encoding Amino Acids (AA)

| AA | Codon | AA | Codons | AA | Codons | AA | Codons |
|---|---|---|---|---|---|---|---|
| Phe (F) | TTT | Ser (S) | TCT | Tyr (Y) | TAT | Cys (C) | TGT |
| | TTC | | TCC | | TAC | | TGC |
| Leu (L) | TTA | | TCA | TERM | TAA | TERM | TGA |
| | TTG | | TCG | | TAG | Trp (W) | TGG |
| | CTT | Pro (P) | CCT | His (H) | CAT | Arg (R) | CGT |
| | CTC | | CCC | | CAC | | CGC |
| | CTA | | CCA | Gln (Q) | CAA | | CGA |
| | CTG | | CCG | | CAG | | CGG |
| Ile (I) | ATT | Thr (T) | ACT | Asn (N) | AAT | Ser (S) | AGT |
| | ATC | | ACC | | AAC | | AGC |
| | ATA | | ACA | Lys (K) | AAA | Arg (R) | AGA |
| Met (M) | ATG | | ACG | | AAG | | AGG |
| Val (V) | GTT | Ala (A) | GCT | Asp (D) | GAT | Gly (G) | GGT |
| | GTC | | GCC | | GAC | | GGC |
| | GTA | | GCA | Glu (E) | GAA | | GGA |
| | GTG | | GCG | | GAG | | GGG |

Exemplary nucleotide (e.g., cDNA) sequences encoding the variable heavy (V_{H}) and variable light chain (V_{L}) amino acid sequences are shown below:

(SEQ ID NO: 57 (10C8 V_{H}))
CAGGTTCAGCTGGTGCAGTCTGGCGCTGAGGTGAAGAAGCCTGGGGCCTCAGTGAA
GGTCTCCTGCAAGGCTTCTGGTTACACCTTAACCAGCTATGGTATCAGCTGGGTGCG
ACAGGCCCCTGGACAAGGTCTTGAGTGGATGGGATGGATCAGCGCTTACAATGGTAA
CACAAATTATGCACAGAAGCTCCAGGGCAGAGTCACCATGACCACAGACACATCCAC
GAGCACAGCCTACATGGAGCTGAGGAGCCTGAGATCTGACGACACGGCCGTGTATT
ATTGTGCGAGAGAGTTATTATGGTTCGGGGAGTTATTATCCCAATACTATTACTACGG
TATGGACGTCTGGGGCCAAGGGACCACGGTCACCGTCTCCTCAG;
(SEQ ID NO: 58 (10C8 V_{L}))
GACATCCAGATGACCCAGTCTCCTTCCACCCTGTCTGCATCTGTAGGAGACAGAGTC
ACCATCACTTGCCGGGCCAGTCAAAATATTAGTCGCTGGTTGGCCTGGTATCAGCAG
AAACCAGGGAAAGCCCCTAAACTCCTGATCTTTAGGGCATCTAGTTTAGAAACTGGG
GTCCCATCAAGGTTCGGCGGCAGTGGATCTGGGACAGAATTCACTCTCACCATCAGC
AGCCTGCAGCCTGATGATTTTGCAACTTATTACTGCCAACAGTATAATAGTTATTACAC
TTTTGGCCAGGGGACCAGGCTGGAGATCAAAC;
(SEQ ID NO: 59 (16C11 V_{H}))
CAGGTGCAGCTGGTGGAGTCTGGGGGAGGCGTGGTCCAGCCTGGGAGGTCCCTGA
GACTCTCCTGTGCAGCGTCTGGATTCACCTTCAGTAGTTATGCCATGCACTGGGTCC
GCCAGGCTCCAGGCAAGGGGCTGGAGTGGGTGGCAGTTATATGGTATGATGGAAGT
AAAAAATACAATGCAGACTCCGTGAAGGGCCGATTCACCATCTCCAGAGACAATTCCA
AGAACACGCTGTATCTGCAAATGAACAGCCTGAGAGCCGAGGACACGGCTGTGTATT
ACTGTGCGAGAGATAGGGGGATAGGTGGGGACTACTGGGGCCAGGGAACCCTGGT
CACCGTCTCCTCAG;
(SEQ ID NO: 60 (16C11 V_{L}))
GACATCGTGATGACCCAGTCTCCAGACTCCCTGGCTGTGTCTCTGGGCGAGAGGGC
CACCATCAACTGCAAGTCCAGTCAGACTGTTTTATACAGCTCCAACAATAAGAACTAC
TTAGCTTGGTACCAGCAGAAGCCAGGACAGCCTCCTAAGCTGCTCATTTACTGGGCA
TCTACCCGGGAATCCGGGGTCCCTGACCGATTCAGTGGCGGCGGGTCTGGGACAGA
TTTCACTCTCACCATCAGCAGCCTGCAGGCTGAAGATGTGGCAGTTTATTACTGTCAA
CAATATTATAGTACTCCGTACACTTTTGGCCAGGGGACCAAGCTGGAGATCAACC;
(SEQ ID NO: 61 (14A6 V_{H}))
CAGGTGCAGCTGGTGGAGTCTGGGGGAGGCGTGGTCCAGCCTGGGAGGTCCCTGA
GACTCTCCTGTGCAGCGTCTGGATTCACCTTCAGTAACTATGGCATGCACTGGGTCC
GCCAGGCTCCAGGCAAGGGGCTGGAGTGGGTGGCAGTTATATGGTATGATGGAAGT
AATAAATACTATGCAGACTCCGTGAAGGGCCGCTTCACCATCTCCAGAGACAATTCCA
AGAACACGCTGTATCTGCAAATGAACAGCCTGAGAGCCGAGGACACGGCTGTCTATT
ACTGTGCGAGAGGGCACTCTGTTTCGGGGGGTGACTACTGGGGCCAGGGAACCCTG
GTCACCGTCTCCTCA;
(SEQ ID NO: 62 (14A6 V_{L}))
GACATCCAGATGACCCAGTCTCCATCCTCCCTGTCTGCATCTGTAGGAGACAGAGTC
ACCATCACTTGCCGGGCAAGTCAGAGCGTGAACAGCTTCTTGAATTGGTATCAGCAG
ACACCAGGGAAAGCCCCTAAACTCCTGATCTATGCTGCATCCAGTTTGCAAAGTGGG
GTCCCACCAAGGTTCAGTGGCAGTGGATCTGGGACAGATTTCACTCTCACCATCAGC
AGTCTGCAACCTGAAGATTTTGCAACTTACTACTGTCAACAGAGTTACAGTACCCCGT
ACACTTTTGGCCAGGGGACCAAGCTGGAGATCAAC;
(SEQ ID NO: 63 (10B3 V_{H}))
CAGGTGCAGCTGGTGGAGTCTGGGGGAGGCGTGGTCCAGCCTGGGAGGTCCCTGA
GACTCTCCTGTGCAGCGTCTGGATTCACCCTCAGTGGCTATGGCATGCACTGGGTCC
GCCAGGCTCCAGACAAGGGGCTGGAGTGGGTGGCAGTTATATGGTATGATGGAAGT
GATAAAAACTATGCAGACTCCGTGAAGGGCCGATTCACCATCTCCAGAGACAATTCCA
AGAACACGCTGTATCTGCAAATGAACAGCCTGAGAGCCGAGGACACGGCTGTGTATT
ACTGTGCGAGAGATCGAGGAGCAGTGGCTGGTGACTACTGGGGCCAGGGAACCCTG
GTCACCGTCTCCTCAG;
and,
(SEQ ID NO: 64 (10B3 V_{L}))
GACATCGTGATGACCCAGTCTCCAGACTCCCTGGCTGTGTCTCTGGGCGAGAGGGC
CACCATCAACTGCAAGTCCAGCCAGAGTGTTTTATACAACTCCAACAATAAGAACCTC
TTAGCCTGGTTCCGGCAGAAACCGGGACAGCCTCCTAAGTTGCTCATTTACTGGGCG
TCTACCCGGGAATCCGGGGTCCCTGACCGGTTCAGTGGCAGCGGGTCTGGGACAGA
TTTCACTCTCACCATCAGCAGCCTGCAGGCTGAAGATGTGGCAGTTTATTTCTGTCAG
CAATATTATGGTACTCCGTACACTTTTGGCCAGGGGACCAAGCTGGAGATCAAAC.

Variations of these nucleotide sequences are also contemplated herein. Compositions comprising the same are also contemplated. Such variations encode antibodies, or polypeptides comprising the CDRs or variants thereof disclosed herein or as would be recognized by those of ordinary skill in the art. Those of ordinary skill in the art understand that the nucleotide sequence encoding a particular amino acid sequence of an equivalent to an 10C8, 16C11, 14A6, or 10B3 antibody may be easily derived from the amino acid sequence of any of SEQ ID NOS. 57-64 and the information presented in Table 3. For instance, it may be deduced from the amino acid sequence GYTLTSY (SEQ ID NO:1) and the information presented in Table 3 that the amino acid sequence may be encoded by the nucleotide sequence GGC TAC ACC CTG ACC AGC TAC (SEQ ID NO: 65). Those of ordinary skill in the art would understand that nucleotide sequences encoding SEQ ID NOS. 1-56 and derivatives thereof may be deduced in the same way, and such nucleotide sequences are contemplated herein. This disclosure also provides an expression vector including an isolated nucleic acid comprising and/or consisting of such nucleotide sequences (in preferred embodiments any of SEQ ID NOS. 57-64 or derivatives thereof) as well as host cells (e.g., a cell line) containing such an expression vector.

The term “operably linked” refers to a functional relationship between two or more polynucleotide (e.g., DNA) segments. Typically, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “recombinant host cell” (or simply “host cell”) or “cell line” refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” or “cell line” as used herein.

The term “subject” includes human and non-human animals. Non-human animals include all vertebrates (e.g.: mammals and non-mammals) such as, non-human primates (e.g.: cynomolgus monkey), sheep, dog, cow, chickens, amphibians, and reptiles. Except when noted, the terms “patient” or “subject” are used herein interchangeably.

As used herein, the term “treating” or “treatment” of any disease or disorder (e.g., breast cancer) refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder. “Prevention” as it relates to indications described herein, including, conditions or disorders associated with cancers that express progranulin.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, it is intended to include such other forms of expression vectors, such as viral vectors (e.g., replication defective retroviruses, adenoviruses and adeno-associated viruses), which serve equivalent functions.

Antibodies can be conjugated with drugs to form antibody-drug conjugates (ADCs). Typically, the ADC contains a linker between the drug and the antibody. The linker can be a degradable or a non-degradable linker. Degradable linkers are typically easily degraded in the intracellular environment, for example, the linker is degraded at the target site, so that the drug is released from the antibody. Suitable degradable linkers include, for example, enzymatically degraded linkers, including peptidyl-containing linkers that can be degraded by intracellular proteases (such as lysosomal proteases or endosomal proteases), or sugar linkers, for example, a glucuronide-containing linker that can be degraded by glucuronidase. The peptidyl linker may include, for example, dipeptides such as valine-citrulline, phenylalanine-lysine or valine-alanine. Other suitable degradable linkers include, for example, pH-sensitive linkers (for example, linkers that are hydrolyzed at a pH of less than 5.5, such as hydrazone linkers) and linkers that degrade under reducing conditions (for example, disulfide bond linkers). Non-degradable linkers typically release the drug under conditions where the antibody is hydrolyzed by a protease.

Before being connected to the antibody, the linker has a reactive group capable of reacting with certain amino acid residues, and the connection is achieved through the reactive group. Sulfhydryl-specific reactive groups are preferred and include, for example, maleimide compounds, halogenated amides (such as iodine, bromine, or chloro); halogenated esters (such as iodine, bromine, or chloro); halogenated methyl ketones (such as iodine, bromine or chloro), benzyl halides (such as iodine, bromine or chloro); vinyl sulfone, pyridyl disulfide; mercury derivatives such as 3,6-Di-(mercury methyl) dioxane, and the counter ion is acetate, chloride or nitrate; and polymethylene dimethyl sulfide thiosulfonate. The linker may include, for example, maleimide linked to the antibody via thiosuccinimide. The drug can be any cytotoxic, inhibiting cell growth or immunosuppressive drug. In embodiments, the linker connects the antibody and the drug, and the drug has a functional group that can be bonded to the linker. For example, the drug may have an amino group, a carboxyl group, a sulfhydryl group, a hydroxyl group, or a ketone group that can form a bond with the linker. In the case where the drug is directly connected to the linker, the drug has a reactive active group before being connected to the antibody. Useful drug categories include, for example, anti-tubulin drugs, DNA minor groove binding reagents, DNA replication inhibitors, alkylating reagents, antibiotics, folate antagonists, antimetabolites, chemotherapy sensitizers, topoisomerase inhibitors, Vinca Alkaloids, etc. Typical cytotoxic drugs include, for example, auristatins, camptothecins, duocarmycins, etoposides, maytansines and maytansinoids (e.g., DM1 and DM4), taxanes, benzodiazepines or benzodiazepine containing drugs (e.g., pyrrolo[1,4]benzodiazepines (PBDs), indolinobenzodiazepines and oxazolidinobenzodiazepines and vinca alkaloids.

As described herein, the drug-linker can be used to form ADC in one simple step. In other embodiments, bifunctional linker compounds can be used to form ADCs in a two-step or multi-step process. For example, the cysteine residue reacts with the reactive part of the linker in the first step, and in the subsequent step, the functional group on the linker reacts with the drug to form ADC. Generally, the functional group on the linker is selected to facilitate the specific reaction with the appropriate reactive group on the drug moiety. As a non-limiting example, the azide-based moiety can be used to specifically react with the reactive alkynyl group on the drug moiety. The drug is covalently bound to the linker through the 1,3-dipolar cycloaddition between the azide and alkynyl groups. Other useful functional groups include, for example, ketones and aldehydes (suitable for reacting with hydrazides and alkoxyamines), phosphines (suitable for reacting with azides); isocyanates and isothiocyanates (suitable for reaction with amines and alcohols); and activated esters, such as N-hydroxysuccinimide ester (suitable for reaction with amines and alcohols). These and other ligation strategies, such as those described in “Bioconjugation Technology”, Second Edition (Elsevier), are well known to those skilled in the art. Those skilled in the art can understand that for the selective reaction between the drug moiety and the linker, when a complementary pair of reactive functional groups is selected, each member of the complementary pair can be used for both linkers and drugs.

The antibodies can be used as a treatment for cancer (e.g., breast cancer, including triple negative breast cancer [TNBC]) or other diseases which exhibit an increased expression of progranulin. Triple negative breast cancer is when the cancer cells do not have the receptors for estrogen, progesterone and the HER2 protein. By the term “neutralizing” it shall be understood that the antibody has the ability to inhibit or block any biological activity of progranulin that leads to tumorigenesis, including its ability to stimulate cell proliferation or to induce tumor growth in experimental animals and in humans. An effective amount of anti-progranulin antibody is administered to a mammal, including humans, by various routes.

In some preferred embodiments, this disclosure provides that is an isolated monoclonal antibody, such as a human monoclonal antibody, that binds PGRN (preferably hu PGRN), and is preferably antibody 10C8, 16C11, 14A6, or 10B3 (which may be used in combination). In some preferred embodiments, the antibody is derived from a human antibody, human IgG, human IgG1, human IgG2, human IgG2a, human IgG2b, human IgG3, human IgG4, human IgM, human IgA, human IgA1, human IgA2, human IgD, human IgE, canine antibody, canine IgGA, canine IgGB, canine IgGC, canine IgGD, chicken antibody, chicken IgA, chicken IgD, chicken IgE, chicken IgG, chicken IgM, chicken IgY, goat antibody, goat IgG, mouse antibody, mouse IgG, pig antibody, rat antibody, Ilaman antibody, alpacan antibody, shark antibody and a camel antibody. In some preferred embodiments, this disclosure provides a derivative of an antibody disclosed herein, optionally selected from the group consisting of an F_{ab}, F_{ab2}, Fab′ single chain antibody, Fv, single chain, mono-specific antibody, bispecific antibody, trimeric antibody, multi-specific antibody, multivalent antibody, chimeric antibody, canine-human chimeric antibody, canine-mouse chimeric antibody, antibody comprising a canine Fc, humanized antibody, human antibody, caninized antibody, CDR-grafted antibody, shark antibody, and a nanobody.

In some preferred embodiments, this disclosure provides a derivative of an antibody disclosed herein comprising a detectable label fixably attached thereto, optionally wherein the detectable label is selected from the group consisting of fluorescein, DyLight, Cy3, Cy5, FITC, HiLyte Fluor 555, HiLyte Fluor 647, 5-carboxy-2,7-dichlorofluorescein, 5-carboxyfluorescein, 5-FAM, hydroxy tryptamine, 5-hydroxy tryptamine (5-HAT), 6-carboxyfluorescein (6-FAM), FITC, 6-carboxy-1,4-dichloro-2′,7′-dichlorofluorescein (TET), 6-carboxy-1,4-dichloro-2′,4′,5′,7′-tetrachlorofluorescein (HEX), 6-carboxy-4′,5′-dichloro-2′,7′-dimethoxyfluorescein (6-JOE), an Alexa fluor, Alexa fluor 350, Alexa fluor 405, Alexa fluor 430, Alexa fluor 488, Alexa fluor 500, Alexa fluor 514, Alexa fluor 532, Alexa fluor 546, Alexa fluor 555, Alexa fluor 568, Alexa fluor 594, Alexa fluor 610, Alexa fluor 633, Alexa fluor 635, Alexa fluor 647, Alexa fluor 660, Alexa fluor 680, Alexa fluor 700, Alexa fluor 750, a BODIPY fluorophores, BODIPY 492/515, BODIPY 493/503, BODIPY 500/510, BODIPY 505/515, BODIPY 530/550, BODIPY 542/563, BODIPY 558/568, BODIPY 564/570, BODIPY 576/589, BODIPY 581/591, BODIPY 630/650-X, BODIPY 650/665-X, BODIPY 665/676, FL, FL ATP, FI-Ceramide, R6G SE, TMR, TMR-X conjugate, TMR-X, SE, TR, TR ATP, TR-X SE, a rhodamine, rhodamine 110, rhodamine 123, rhodamine B, rhodamine B 200, rhodamine BB, rhodamine BG, rhodamine B extra, 5-carboxytetramethylrhodamine (5-TAMRA), 5 GLD, 6-carboxyrhodamine 6G, Lissamine, Lissamine Rhodamine B, Phallicidine, Phalloidine, rhodamine red, Rhod-2, 6-carboxy-X-rhodamine (ROX), carboxy-X-rhodamine (5-ROX), Sulphorhodamine B can C, Sulphorhodamine G Extra, 6-carboxytetramethylrhodamine (TAMRA), tetramethylrhodamine (TRITC), rhodamine WT, Texas Red, and Texas Red-X.

In some preferred embodiments, this disclosure provides a derivative of an antibody disclosed herein comprising an effector moiety attached thereto, optionally wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical, optionally comprising a cleavable linker positioned between the antibody and the effector moiety, wherein said cleavable linker releases the effector moiety into or within a cell. In some preferred embodiments, this disclosure provides an isolated polynucleotide encoding an antibody of this disclosure, optionally wherein a nucleic acid sequence is of those disclosed herein, as well as an expression vector and host cells comprising the same. In some preferred embodiments, this disclosure provides a composition comprising at least antibody or derivative of disclosed herein; at least one isolated polynucleotide encoding such an antibody or derivative; or at least one expression vector comprising such a polynucleotides; and/or, at least one host cell comprising such a polynucleotide and/or expression vector; or a combination thereof; and, a pharmaceutically acceptable carrier.

In some preferred embodiments, this disclosure provides methods for detecting PGRN on a cell, the method comprising contacting a test biological sample with an antibody or derivative of this disclosure and detecting the antibody bound to the biological sample or components thereof. In some embodiments, the methods comprise comparing the amount of binding to the test biological sample or components thereof to the amount of binding to a control biological sample or components thereof, wherein increased binding to the test biological sample or components thereof relative to the control biological sample or components thereof indicates the presence of a cell expressing PGRN (preferably hu PGRN) in the test biological sample (e.g., wherein the test biological sample is a mammalian cell, tissue, or blood). The method may be in vivo method or an in vitro method.

In some preferred embodiments, this disclosure provides methods for treating, preventing and/or ameliorating cancer in a mammal comprising administering to the mammal at least one effective dose of a pharmaceutical composition comprising an antibody or derivative of this disclosure. In some embodiments, this disclosure provides such an antibody comprising a cytotoxic effector moiety attached thereto, optionally wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical. In some embodiments, such antibodies can comprise a cleavable linker positioned between the antibody and the effector moiety, wherein said cleavable linker releases the effector moiety into or within a cell. In some such embodiments, the antibody is administered as an antibody-drug conjugate. In some embodiments, multiple doses are administered to the animal; and/or, the antibody is administered in a dosage amount of about 1 to 50 mg/kg.

The antibodies (e.g., polypeptides) and nucleic acids described herein may also be combined with one or more pharmaceutically acceptable carriers prior to administration to a host. A pharmaceutically acceptable carrier is a material that is not biologically or otherwise undesirable, e.g., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Suitable pharmaceutical carriers and their formulations are described in, for example, Remington's: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those persons skilled in the art that certain carriers may be more preferable depending upon, for instance, the route of administration and concentration of composition being administered. Carriers are those suitable for administration of polypeptides and/or fragments thereof to humans or other subjects. Pharmaceutical compositions may also include carriers, thickeners, diluents, buffers, preservatives, surface active agents, adjuvants, immunostimulants, in addition to the immunogenic polypeptide. Pharmaceutical compositions may also include one or more active ingredients such as antimicrobial agents, anti-inflammatory agents and anesthetics. The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of a nucleic acid or polypeptide. The terms “effective amount” and “therapeutically effective amount” each refer to the amount of an antibody, nucleic acid or the like used to observe the desired therapeutic effect (e.g., eliminating PGRN-expressing cells, e.g., cancerous PGRN-expressing cells).

Methods for treating one or more disease conditions (e.g., cancer) in a mammalian host comprising administering to the mammal at least one or more effective doses of one or more antibodies (and/or derivative(s) thereof) described herein are also provided. In some embodiments, the antibody is a monoclonal antibody or fragment or derivative thereof comprising one or more of the combinations of CDRs and/or variable regions of antibody 10C8, 16C11, 14A6, or 10B3; the amino acid sequences shown in Table 1 and/or encoded by a nucleotide sequence disclosed herein or as may be recognized by one of ordinary skill in the art; and/or substituted derivatives and/or fragments thereof; as well as in some embodiments conservatively substituted variants thereof. The one or more antibodies may be administered in a dosage amount of about 1 to about 50 mg/kg, about 1 to about 30 mg/kg, or about 5 to about 30 mg/kg (e.g., about any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 mg/kg). In certain embodiments, the one or more antibodies may be administered to the mammal (e.g., intradermally, intravenously, orally, rectally) at about 10 mg/kg one or more times. When multiple doses are administered, the doses may comprise about the same or different amount of antibody in each dose. The doses may also be separated in time from one another by the same or different intervals. For instance, the doses may be separated by about any of 6, 12, 24, 36, 48, 60, 72, 84, or 96 hours, one week, two weeks, three weeks, one month, two months, three months, four months, five months, six months, seven months, eight months, nine months, 10 months, 11 months, 12 months, 1.5 years, 2 years, 3 years, 4 years, 5 years, or any time period before, after, and/or between any of these time periods. In some embodiments, the antibodies may be administered in conjunction with other agents (e.g., anti-infective agents and/or chemotherapeutic agent). Such other agents may be administered about simultaneously with the antibodies, or at a different time and/or frequency. Other embodiments of such methods may also be appropriate as could be readily determined by one of ordinary skill in the art.

In some preferred embodiments, this disclosure provides an isolated antibody or antigen binding fragment thereof, comprising: a) a heavy chain variable region comprising the CDR sequences SEQ ID NOs: 1, 2, and 3 and a light chain variable region comprising CDR sequences SEQ ID NOs: 4, 5, and 6, respectively; b) a heavy chain variable region comprising the CDR sequences SEQ ID NOs: 7, 8, and 9 and a light chain variable region comprising CDR sequences SEQ ID NOs: 10, 11 and 12, respectively; c) a heavy chain variable region comprising the CDR sequences SEQ ID NOs: 13, 14, and 15 and a light chain variable region comprising CDR sequences SEQ ID NOs: 16, 17, and 18, respectively; d) a heavy chain variable region comprising the CDR sequences SEQ ID NOs: 19, 20, 21 and a light chain variable region comprising CDR sequences SEQ ID NOs: 22, 23, and 24, respectively; e) heavy chain and light chain variable regions comprising the CDR sequences, respectively, SEQ ID NOS. 1-3 and 7-9, respectively; f) heavy chain and light chain variable regions comprising the CDR sequences, respectively, SEQ ID NOS. 4-6 and 10-12, respectively; g) heavy chain and light chain variable regions comprising, respectively, SEQ ID NO. 13 and SEQ ID NO. 14, respectively; h) heavy chain and light chain variable regions comprising the CDR sequences, respectively, SEQ ID NOS. 15-17 and 21-23, respectively; i) heavy chain and light chain variable regions comprising the CDR sequences, respectively, SEQ ID NOS. 18-20 and 24-26, respectively; j) heavy chain and light chain variable regions comprising SEQ ID NO. 27 and SEQ ID NO. 28, respectively; k) heavy chain and light chain variable regions comprising the CDR sequences, respectively, SEQ ID NOS. 29-31 and 35-37, respectively; l) heavy chain and light chain variable regions comprising the CDR sequences SEQ ID NOS. 32-34 and 38-40, respectively; m) heavy chain and light chain variable regions comprising, respectively, SEQ ID NO. 41 and SEQ ID NO. 42, respectively; n) heavy chain and light chain variable regions comprising the CDR sequences SEQ ID NOS. 43-45 and 49-51, respectively; o) heavy chain and light chain variable regions comprising the CDR sequences SEQ ID NOS. 46-48 and 52-54, respectively; p) heavy chain and light chain variable regions comprising SEQ ID NO. 55 and SEQ ID NO. 56, respectively; or a derivative of any one of a)-p); wherein the antibody or antigen binding fragment thereof specifically binds to human progranulin. In some preferred embodiments, this disclosure provides an isolated antibody or antigen binding fragment thereof, comprising: a) a heavy chain variable region comprising SEQ ID NO: 25 and a light chain variable region comprising SEQ ID NO:27, respectively; b) a heavy chain variable region comprising SEQ ID NO: 29 and a light chain variable region comprising SEQ ID NO:31, respectively; c) a heavy chain variable region comprising SEQ ID NO: 33 and a light chain variable region comprising SEQ ID NO:35, respectively; or, d) a heavy chain variable region comprising SEQ ID NO: 37 and a light chain variable region comprising SEQ ID NO: 39, respectively. In some preferred embodiments, the antibody is internalized into a cell that expresses hu PGRN in vitro and/or in vivo. In some preferred embodiments, the antibody competes with trastuzumab for binding to hu PGRN receptor on the cell. In some preferred embodiments, the antibody does not compete with trastuzumab for binding to hu PGRN receptor on the cell. In some preferred embodiments, the antibody is 10C8, 16C11, 14A6, and 10B3, or a derivative thereof. In some preferred embodiments, this disclosure provides combinations of such antibodies. In some preferred embodiments, the antibody is an isolated monoclonal antibody. In some preferred embodiments, the antibody is a human monoclonal antibody. In some preferred embodiments, the antibody is derived from a human antibody, human IgG, human IgG1, human IgG2, human IgG2a, human IgG2b, human IgG3, human IgG4, human IgM, human IgA, human IgA1, human IgA2, human IgD, human IgE, canine antibody, canine IgGA, canine IgGB, canine IgGC, canine IgGD, chicken antibody, chicken IgA, chicken IgD, chicken IgE, chicken IgG, chicken IgM, chicken IgY, goat antibody, goat IgG, mouse antibody, mouse IgG, pig antibody, rat antibody, Ilaman antibody, alpacan antibody, shark antibody and a camel antibody. In some preferred embodiments, the derivative of the antibody is selected from the group consisting of an F_{ab}, F_{ab2}, Fab′ single chain antibody, Fv, single chain, mono-specific antibody, bispecific antibody, trimeric antibody, multi-specific antibody, multivalent antibody, chimeric antibody, canine-human chimeric antibody, canine-mouse chimeric antibody, antibody comprising a canine Fc, humanized antibody, human antibody, caninized antibody, CDR-grafted antibody, shark antibody, and a nanobody. In some preferred embodiments, the antibody or derivative comprises a detectable label fixably attached thereto, optionally wherein the detectable label is selected from the group consisting of fluorescein, DyLight, Cy3, Cy5, FITC, HiLyte Fluor 555, HiLyte Fluor 647, 5-carboxy-2,7-dichlorofluorescein, 5-carboxyfluorescein, 5-FAM, hydroxy tryptamine, 5-hydroxy tryptamine (5-HAT), 6-carboxyfluorescein (6-FAM), FITC, 6-carboxy-1,4-dichloro-2′,7′-dichlorofluorescein (TET), 6-carboxy-1,4-dichloro-2′,4′,5′,7′-tetrachlorofluorescein (HEX), 6-carboxy-4′,5′-dichloro-2′,7′-dimethoxyfluorescein (6-JOE), an Alexa fluor, Alexa fluor 350, Alexa fluor 405, Alexa fluor 430, Alexa fluor 488, Alexa fluor 500, Alexa fluor 514, Alexa fluor 532, Alexa fluor 546, Alexa fluor 555, Alexa fluor 568, Alexa fluor 594, Alexa fluor 610, Alexa fluor 633, Alexa fluor 635, Alexa fluor 647, Alexa fluor 660, Alexa fluor 680, Alexa fluor 700, Alexa fluor 750, a BODIPY fluorophores, BODIPY 492/515, BODIPY 493/503, BODIPY 500/510, BODIPY 505/515, BODIPY 530/550, BODIPY 542/563, BODIPY 558/568, BODIPY 564/570, BODIPY 576/589, BODIPY 581/591, BODIPY 630/650-X, BODIPY 650/665-X, BODIPY 665/676, FL, FL ATP, FI-Ceramide, R6G SE, TMR, TMR-X conjugate, TMR-X, SE, TR, TR ATP, TR-X SE, a rhodamine, rhodamine 110, rhodamine 123, rhodamine B, rhodamine B 200, rhodamine BB, rhodamine BG, rhodamine B extra, 5-carboxytetramethylrhodamine (5-TAMRA), 5 GLD, 6-carboxyrhodamine 6G, Lissamine, Lissamine Rhodamine B, Phallicidine, Phalloidine, rhodamine red, Rhod-2, 6-carboxy-X-rhodamine (ROX), carboxy-X-rhodamine (5-ROX), Sulphorhodamine B can C, Sulphorhodamine G Extra, 6-carboxytetramethylrhodamine (TAMRA), tetramethylrhodamine (TRITC), rhodamine WT, Texas Red, and Texas Red-X. In some preferred embodiments, the antibody or derivative comprises an effector moiety attached thereto, optionally wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical. In some preferred embodiments, the antibody or derivative can comprise a cleavable linker positioned between the antibody and the effector moiety, wherein said cleavable linker releases the effector moiety into or within a cell. In some preferred embodiments, this disclosure provides an isolated polynucleotide encoding an antibody disclosed herein, optionally wherein a nucleic acid sequence of at least one of SEQ ID NOS. 57-64. In some preferred embodiments, the polynucleotide can be an expression vector comprising one or more such polynucleotides. In some preferred embodiments, this disclosure provides a host cell comprising such isolated polynucleotide(s) and/or the expression vector(s). In some preferred embodiments, this disclosure provides a composition comprising any such at least antibody and/or derivative; at least one isolated polynucleotide encoding the same; at least one expression vector comprising and/or encoding the same; and/or, at least one host cell of comprising the same; or a combination of any thereof; and, a pharmaceutically acceptable carrier.

In some preferred embodiments, this disclosure provides methods=for detecting hu PGRN (GP88) on a cell, the method comprising contacting a test biological sample with an antibody or derivative of this disclosure and detecting the same bound to the biological sample or components thereof. In some preferred embodiments, the cell can be selected from the group consisting of a breast cancer, adenocarcinoma epidermal growth factor receptor (EGF-R) breast cancer, basal breast carcinoma, estrogen receptor (ER) luminal breast cancer, ER positive tamoxifen resistant luminal breast cancer, letrozole resistant luminal breast cancer, biliary cancer, bladder cancer, brain cancer, glioblastoma, colorectal cancer, epidermoid carcinoma, squamous carcinoma, esophogeal cancer, gastric cancer, hepatocellular cancer, kidney/renal cancer, laryngeal cancer, lung cancer, non-small-cell lung carcinoma, lung adenocarcinoma, mesothelioma, myeloma/leukemia, ovarian cancer, prostate cancer, and uterine cancer cell. In some preferred embodiments, the methods can comprise=comparing the amount of binding to the test biological sample or components thereof to the amount of binding to a control biological sample or components thereof, wherein increased binding to the test biological sample or components thereof relative to the control biological sample or components thereof indicates the presence of a cell expressing hu PGRN in the test biological sample. In some preferred embodiments, the test biological sample can be a mammalian cell, tissue, or blood. In some preferred embodiments, the method is an in vivo method or an in vitro method.

In some preferred embodiments, this disclosure provides methods for treating, preventing and/or ameliorating cancer in a mammal comprising administering to the mammal at least one effective dose of a pharmaceutical composition comprising an antibody or derivative of this disclosure. In some preferred embodiments, the cancer is selected from the group consisting of breast cancer, adenocarcinoma epidermal growth factor receptor (EGF-R) breast cancer, basal breast carcinoma, estrogen receptor (ER) luminal breast cancer, ER positive tamoxifen resistant luminal breast cancer, letrozole resistant luminal breast cancer, biliary cancer, bladder cancer, brain cancer, glioblastoma, colorectal cancer, epidermoid carcinoma, squamous carcinoma, esophogeal cancer, gastric cancer, hepatocellular cancer, kidney/renal cancer, laryngeal cancer, lung cancer, non-small-cell lung carcinoma, lung adenocarcinoma, mesothelioma, myeloma/leukemia, ovarian cancer, prostate cancer, and uterine cancer. In some preferred embodiments, the antibody comprises a cytotoxic effector moiety attached thereto, optionally wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical. In some preferred embodiments, the antibody further comprises a cleavable linker positioned between the antibody and the effector moiety, wherein said cleavable linker releases the effector moiety into or within a cell. In some preferred embodiments, the antibody is administered as an antibody-drug conjugate. In some preferred embodiments, multiple doses are administered to the animal; and/or, the antibody is administered in a dosage amount of about 1 to 50 mg/kg.

In some preferred embodiments, this disclosure provides a kit for detecting the expression of hu PGRN (GP88) in or on a cell, the kit comprising an antibody or derivative of any preceding claim and instructions for use. In some preferred embodiments, the cell is selected from the group consisting of a breast cancer, adenocarcinoma epidermal growth factor receptor (EGF-R) breast cancer, basal breast carcinoma, estrogen receptor (ER) luminal breast cancer, ER positive tamoxifen resistant luminal breast cancer, letrozole resistant luminal breast cancer, biliary cancer, bladder cancer, brain cancer, glioblastoma, colorectal cancer, epidermoid carcinoma, squamous carcinoma, esophogeal cancer, gastric cancer, hepatocellular cancer, kidney/renal cancer, laryngeal cancer, lung cancer, non-small-cell lung carcinoma, lung adenocarcinoma, mesothelioma, myeloma/leukemia, ovarian cancer, prostate cancer, and uterine cancer cell. In some preferred embodiments, the antibody or derivative is in lyophilized form.

The following examples related to the monoclonal antibodies described herein.

### Example 1. Discovery and Isolation of High-Affinity Fully Human Monoclonal Antibodies Against Human Progranulin

Immunization of humanized mice TC-mAb™ with human recombinant his-tagged full-length progranulin. The characteristics of these mice are published (Moriwaki, A. et al., Exp Cell Res 390(2): 111914, 2020). Human Ab producing Tc mice (TC-mAb mice) stably maintain a mouse-derived engineered chromosome containing the entire human Ig heavy and kappa chain loci in a mouse Ig knockout background. Trans-chromosomic (Tc) mice carrying mini-chromosomes with human immunoglobulin (Ig) loci can contribute to the development of fully human therapeutic monoclonal antibodies (Abs) when immunized with an antigen of interest. In this case, TC-mAb mice were immunized with human recombinant progranulin. Titer of sera from immunized mice was checked by EIA with human progranulin immobilized to Nickel plates (progranulin) EIA with HRP-conjugated goat anti-human Fc secondary antibody. Mouse with the highest anti-progranulin titer was used to collect spleen and lymph node B cells that were fused by electroporation to mouse myeloma HL-1 cells. Fused hybridomas were single-cell plated in semi-solid hybridoma culture medium D in 10 cm tissue culture plates. After 11 days, 1,726 single hybridoma clones were picked from the semi-solid medium plates and transferred to 96-well dishes (one clone per well) in hybridoma culture medium E. After 3 days, culture media of the hybridoma clones were assayed by progranulin EIA. The assay provided a number of clones. 184 clones had an OD₆₅₀>3.0, 387 clones had an OD₆₅₀ between 3.0 and 2.0, 423 clones had an OD₆₅₀ between 2.0 and 1.0 and 334 clones had an OD₆₅₀ between 1 and 0.5. The top 184 clones with an OD₆₅₀>3.0 were transferred to 48 well plates for confirmatory screening by progranulin EIA as described above. 170 clones were confirmed strong positive. These clones were transferred to 6 well plates in duplicate in medium E. Cells were cryo-preserved in appropriate culture conditions for long-term storage in liquid nitrogen while culture media containing secreted Ig were collected and stored for future evaluation and selection of hybridomas of interest. Out of these clones, 120 antibody producing clones were prepared.

### Example 2. Further Antigen Binding Characterization and Selection of Anti-Progranulin Monoclonal Antibody Clones

In Vitro Proliferation Assays. Culture media from the selected clones were assayed for their ability to inhibit proliferation of TNBC cells MDA-MB-231 by following inhibition of phosphorylation of ERK1/2 (p-ERK1/2) and of AKT (p-AKT), well known signaling molecules in the proliferation survival pathways of cells, including cancer cells. This assay is described in Guha et al, 2021. Using this assay, it was shown that addition of AG01 would inhibit the phosphorylation of ERK1/2 (MAP kinase or MAPK) and of the survival signaling molecule p-AKT in a dose dependent fashion. All the fully human hybridoma clones that were positive by EIA for binding to progranulin were examined in this signaling molecule phosphorylation assay with MDA-MB-231 triple negative breast cancer cells. Based on these assays, 45 clones showed inhibitory activities for p-ERK1/2, p-AKT or both at levels >50% when compared to control cells treated with human IgG as control. Repeated assays further narrowed down the number of clones displaying inhibition of p-ERK1/2 to 38. The results are shown in Table 4.

TABLE 4
Activity of Cloned Monoclonal Antibodies

| Clone | pERK1/2 | pAKT |
|---|---|---|
| CONTROL | 100 | 100 |
| 4F10 | 20 | 10 |
| 6F6 | 100 | 10 |
| 7E 6 | 100 | 10 |
| 7G1 | 90 | 50 |
| 8F7 | 50 | 80 |
| 8G6 | 120 | 50 |
| 8H2 | 200 | 50 |
| 9D2 | 100 | |
| 9E 12 | 80 | |
| 9H3 | 100 | 20 |
| 9G7 | 100 | |
| 9G8 | 80 | |
| 10B1 | 100 | |
| 10B3 | 10 | 50 |
| 10C7 | 10 | 10 |
| 10C8 | 8 | 20 |
| 10D4 | 150 | |
| 10D7 | 120 | |
| 10D8 | 100 | |
| 10F10 | 10 | |
| 10F5 | 10 | |
| 10H12 | 10 | 10 |
| 10H6 | 20 | 20 |
| 10H9 | 100 | |
| 11A11 | 80 | |
| 11B7 | 80 | |
| 11C1 | 90 | 20 |
| 11C10 | 20 | 20 |
| 11D10 | 20 | 50 |
| 11E 2 | 10 | 50 |
| 11E 8 | 300 | |
| 11G7 | 100 | |
| 11G9 | 75 | |
| 11H7 | 100 | |
| 12A3 | 100 | |
| 12A9 | 50 | |
| 12B9 | 150 | |
| 12C3 | 120 | |
| 12D1 | 10 | 50 |
| 12E 12 | 8 | 10 |
| 12E 2 | 10 | 10 |
| 12G1 | 8 | 10 |
| 13A2 | 100 | |
| 13B10 | 75 | |
| 13B7 | 100 | |
| 13D4 | 100 | |
| 13F1 | 300 | |
| 13F2 | 300 | |
| 13G1 | 300 | |
| 13G7 | 50 | 8 |
| 13H2 | 300 | |
| 13H7 | 300 | |
| 13H9 | 200 | |
| 14A6 | 50 | 8 |
| 14A9 | 50 | 20 |
| 14B1 | 100 | |
| 14B5 | 100 | |
| 14B9 | 50 | 50 |
| 14D8 | 20 | 50 |
| 14E 5 | 50 | 20 |
| 1EF12 | 100 | 10 |
| 14F4 | 80 | |
| 14F6 | 200 | |
| 14G4 | 200 | |
| 15a9 | 200 | |
| 15B10 | 100 | |
| 15C5 | 100 | |
| 15C8 | 80 | 50 |
| 15C9 | 200 | |
| 15D1 | 100 | 50 |
| 15D10 | 100 | |
| 15D5 | 200 | |
| 15E 1 | 200 | |
| 15E 5 | 100 | 10 |
| 15F2 | 200 | |
| 15F5 | 100 | |
| 16F6 | 100 | |
| 15G11 | 130 | |
| 15G5 | 200 | |
| 15H1 | 200 | |
| 15H5 | 50 | 200 |
| 15H6 | 50 | 10 |
| 15H8 | 200 | |
| 15H7 | 50 | |
| 16A6 | 100 | |
| 16A8 | 100 | 50 |
| 16B1 | 100 | 50 |
| 16B11 | 100 | |
| 16B2 | 200 | |
| 16B3 | 200 | |
| 16C11 | 80 | 40 |
| 1E 2 | 300 | |
| 16E 4 | 300 | |
| 16G2 | 50 | 10 |
| 16G9 | 200 | |
| 16H1 | 200 | |
| 16H5 | 100 | 40 |
| 16G4 | 300 | |
| 16G5 | 300 | |
| 17B12 | 300 | |
| 17F5 | 300 | |
| 17G12 | 300 | |
| 17G3 | 300 | |
| 18B12 | 300 | |
| 18B5 | 300 | |
| 18B6 | 100 | 50 |
| 18B9 | 300 | |
| 18C1 | 300 | |
| 18C11 | 300 | |
| 18C6 | 100 | 50 |
| 18D1 | 300 | |
| 18D9 | 300 | |
| 18E 1 | 300 | |
| 18E 5 | 300 | |
| 18F12 | 100 | |
| 18F6 | 200 | 10 |
| 18F8 | 200 | |
| 18F8 | 200 | |
| 18G11 | 200 | |
| 18G2 | 200 | |
| 18G5 | 50 | 50 |
| 18G9 | 100 | 10 |

The clones which produced antibodies that inhibited pERK1/2 and/or p-AKT by >50% are highlighted in gray. These 45 clones were then investigated for their ability to inhibit progranulin binding to MDA-MB-231 cells in a dose-dependent fashion.

Progranulin Binding by Flow Cytometry. Binding assays were carried out on an INTELLICYT Flow Cytometer using the instructions included therein. Additional binding assays were conducted, including the ability of the anti-progranulin antibodies to inhibit the binding of progranulin to two cell lines, human embryonic kidney HEK-293 and TNBC MDA-MB-231, and several other cancer cells such as non-small cell carcinoma cell lines H1299. Out of the 45 mAbs assayed, 11 clones (shown in bold in Table 5) produced antibodies that were able to inhibit progranulin binding in a dose dependent fashion.

TABLE 5
Inhibition of Progranulin Flow
Binding on MDA-MB-231 Cells

| | % | | % |
|---|---|---|---|
| Clones | Control | Clones | Control |
| hlgG | 100 | hlgG | 100 |
| c4F10 | 83 | 14A6 | 116 |
| 6F6 | 130 | 14A9 | 115 |
| 7E 6 | 104 | 14B9 | 45 |
| 7G1 | 187 | 14D8 | 140 |
| 8F7 | 120 | 14E 5 | 121 |
| 8G6 | 116 | 14F12 | 149 |
| 8H2 | 145 | 15C8 | 123 |
| 10B3 | 62 | 15D1 | 144 |
| 10C7 | 91 | 15E 5 | 148 |
| 10C8 | 55 | 15H5 | 119 |
| 10F10 | 78 | 15H6 | 71 |
| 10F5 | 76 | 15H7 | 110 |
| 10H12 | 121 | 16A8 | 111 |
| 10H6 | 122 | 16B1 | 135 |
| 11C1 | 129 | 16C11 | 60 |
| 11C10 | 85 | 16G2 | 130 |
| 11D10 | 82 | 16H5 | 138 |
| 11E 2 | 136 | 18B6 | 108 |
| 12D1 | 144 | 18C6 | 125 |
| 12E 12 | 126 | 18F5 | 176 |
| 12E 2 | 155 | 18G5 | 53 |
| 12G1 | 140 | 18G9 | 141 |
| 13G7 | 126 | 9H3 | 48 |

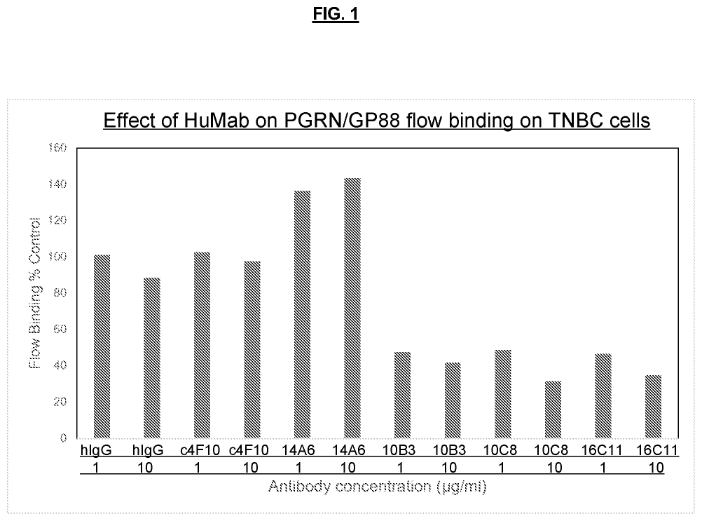

Several antibodies that were able to block p-ERK1/2 in the in vitro assays had little activity in inhibiting progranulin binding, or even stimulated the binding of progranulin to the cells. After further selection by combining several assays, four antibodies were examined. FIG. 1 shows that human anti-progranulin antibodies inhibit the binding of progranulin to TNBC MDA-MB-231 cells in a dose-dependent fashion and have higher activity than AG01, except for 14A6, the addition of which resulted in an increased binding of progranulin in this assay.

Determination of Binding Affinity by Octet. OctetRed96 BLI technology was used to examine the binding characteristics of selected fully human antibodies to progranulin. The method is briefly described herein. Assays were performed at 25° C. Pre-conditioning of the pre hydrated (1 hr) anti-human Fc capture (AHC) sensors was performed by repeating 3 cycles of dipping sensors in regeneration buffer followed by neutralizing buffer (PBS). Anti-progranulin mAbs were captured at 20 μg/ml for 360 seconds on pre-conditioned biosensors. Sensors were then dipped in baseline buffer similar to the progranulin diluent (PBS). Sensors were then dipped into progranulin-containing wells as indicated for 420 seconds. The dissociation step was carried out in assay buffer (PBS) for 600 seconds. The K_{D} was determined based on Kₒₙ and K_{off} using the experimental conditions described above. Table 6 shows the K_{D} of the selected mAbs. In particular, mAbs 16C11, 10B3, 10C8 and 14A6 have a K_{D} of <10⁻⁹ M, with 10C8 having the highest affinity at a K_{D} or 4×10⁻¹² M. These four mAbs were determined to contain IgG1/kappa isotypes based on Iso-Gold Rapid Isotyping.

TABLE 6
K_{D} of Selected Monoclonal Antibodies Using Octet Assay*

| Loading | | | | | Full | Full |
|---|---|---|---|---|---|---|
| mAb ID | Response | K_{D} (M) | Kₒₙ (1/Ms) | K_{off} (1/s) | X² | R² |
| 16C11 | 0.9324 | 4.76 × 10⁻¹⁰ | 1.52 × 10⁵ | 7.21 × 10⁵ | Full X² | Full R² |
| 10B3 | 0.7975 | 1.36 × 10⁻¹⁰ | 8.68 × 10⁴ | 1.19 × 10⁵ | 0.0569 | 0.9961 |
| 10C8 | 0.8898 | 4.13 × 10⁻¹² | 1.30 × 10⁵ | 5.38 × 10⁷ | 0.0078 | 0.9995 |
| 10C7 | 0.6235 | 1.07 × 10⁻⁹ | 8.65 × 10⁴ | 9.28 × 10⁵ | 0.038 | 0.9976 |
| 18G9 | 0.027 | 1.32 × 10⁻⁵ | 7.82 × 10² | 1.04 × 10² | 0.0681 | 0.4423 |
| 14A6 | 0.6397 | 5.87 × 10⁻¹⁰ | 5.03 × 10⁴ | 2.95 × 10⁵ | 0.0014 | 0.9999 |
| 14F12 | 0.9831 | 5.23 × 10⁻⁹ | 1.53 × 10⁵ | 7.98 × 10⁴ | 0.0446 | 0.9967 |

*Sensor type: AHC;
Antibody concentration: 50 nM;
Analyte: huPGRN

Monoclonal Antibody Binding to AG01 Epitope. Experiments were carried out to determine whether any of the selected fully human antibodies competed with AG01 for binding to progranulin, as a way to determine whether these mAbs have similar or overlapping epitopes to those of mAb AG01. This used Bio-Layer Interferometry on an OctetRed96 according to the methods described in Dafferner, A. et al., Chem Res Toxicol 30:1897-1910, 2017. Each of the clones was tested on OctetRed96 for AG01 and test monoclonal co-binding to progranulin in tandem. The assays were performed essentially by capturing either AG01 on AHC sensors or progranulin on HIS1 K sensors and tested for pairing or blocking ability of test antibodies. The results show that the four selected fully human progranulin antibody clones bind to progranulin preoccupied with AG01 without any interference. These data indicate that the four selected antibodies 16C11, 11C8, 10B3 and 14A6 bind to an epitope different from the epitope bound by AG01. These results were confirmed by detailed surface plasmon resonance analysis of AG01 and mAb 16C11 using a Biacore using method described in Myszka, D. et al. (Biophys J 75:583-59, 1998).

### Example 3: Functional Activity of Anti-Progranulin Monoclonal Antibodies

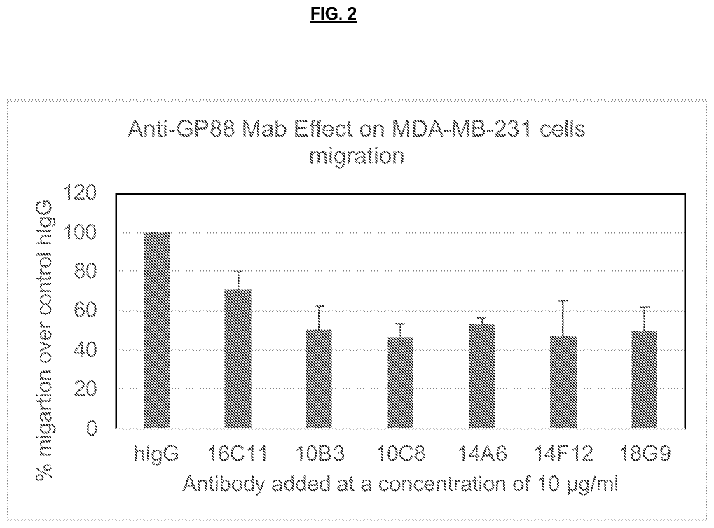

Migration assay. Following the migration assay described in Guha et al., 2021. Eleven clones were examined in the Transwell chamber migration assay, to select the clones exhibiting highest best inhibition of migration of TNBC MDA-MB-231 cells, in a dose dependent fashion in comparison to AG01 as a positive control. Six clones were further retained for their ability to inhibit migration of MDA-MB-231 cells in a dose dependent fashion. The results in FIG. 2 show that these mAbs can inhibit migration of MDA-MB-231 cells in a comparable, if not higher, level than AG01. In particular, the addition of the fully human mAbs at 10 μg/ml inhibited migration of MDA-MB-231 cells by 30 to 50%, while AG01 required a concentration of 50 μg/ml (Guha et al., 2021) for a similar level of inhibition of migration.

### Example 4: Effect of Anti-Progranulin Monoclonal Antibodies on In Vivo Tumorigenesis

These mAbs with the highest affinity (16C11, 10C8, 10B3 and 14A6) were then examined in vivo for their ability to block tumor growth of MDA-MB-231 cells injected subcutaneously in the flank of female athymic nude mice. It has been shown that progranulin expression is associated with increased tumorigenesis (Tangkeangsirisin, Hayashi et al., 2004) and that mice bearing TNBC tumors exhibited tumor growth inhibition when treated with anti-progranulin antibody AG01 (Guha, et al., 2021). Thus, we examined the ability of fully human anti-progranulin antibodies to inhibit tumor growth of the TNBC cell line MDA-MB-231 using the methods described in Guha, et al., 2021. Three antibodies were tested in these nude mice. All antibodies were assayed at a concentration of 5 mg/kg. Athymic female mice were injected subcutaneously with 106 MDA-MB-231 cells. When the tumors reached 100 mm³, the mice were randomized to the various experimental groups with seven animals per group. Antibodies were injected twice weekly intraperitoneally at a dose of 5 mg/kg. Tumor size was determined using a caliper twice weekly before injection of the antibodies, and tumor volume was determined from the dimensions of the tumor.

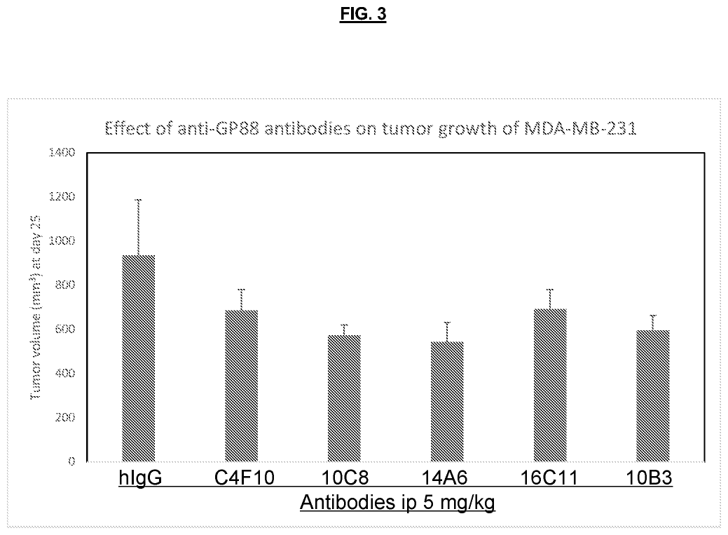

FIG. 3 shows the effect of intraperitoneal injection of fully human antibodies on tumor volume determined at day 25 of treatment when compared to AG01 positive control and human IgG negative control. The data show that the selected antibodies displayed various degrees of tumor inhibition when compared to AG01 used as positive control. These data indicate that the four selected antibodies are able to inhibit tumor growth of a TNBC cell line with similar or higher efficacy than mAb A01.

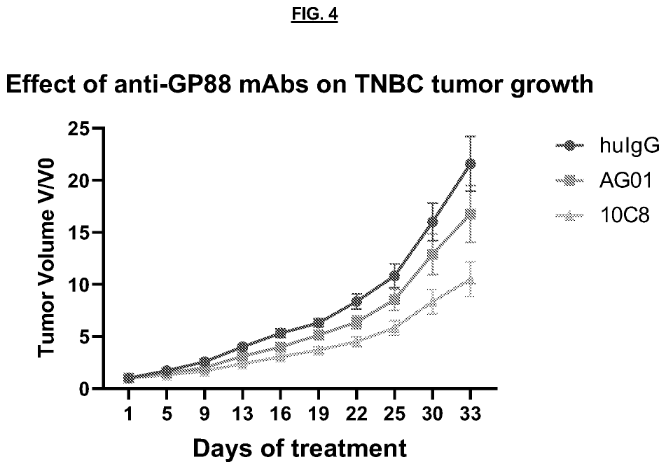

Based on these data and the binding data for mAb 10C8, a further comparison of the anti-tumor activity of mAbs 10C8 and AG01 was performed throughout the treatment course. The graph in FIG. 4 shows tumor growth inhibition by 10C8 throughout the course of treatment. 10C8 has a two-fold higher tumor inhibition activity than AG01 at the same dose of 5 mg/kg (P<0.006). The tumor growth was inhibited by 50% with twice weekly injection of 10C8 when compared to human IgG as control.

Other embodiments will be apparent to those skilled in the art from consideration of the specification and directions provided herein. It is intended that the specification and examples be considered as exemplary only, with a true scope and spirit herein being indicated by the following claims.

1-33. (canceled)

34. An isolated antibody or antigen binding fragment thereof, comprising:
a) a heavy chain variable (V_{H}) region comprising the CDR sequences SEQ ID NOs: 1, 2, and 3 and a light chain variable (V_{L}) region comprising CDR sequences SEQ ID NOs: 4, 5, and 6, respectively;
b) a heavy chain variable (V_{H}) region comprising the CDR sequences SEQ ID NOs: 7, 8, and 9 and a light chain variable region comprising CDR sequences SEQ ID NOs: 10, 11 and 12, respectively;
c) a heavy chain variable (V_{H}) region comprising the CDR sequences SEQ ID NOs: 13, 14, and 15 and a light chain variable (V_{L}) region comprising CDR sequences SEQ ID NOs: 16, 17, and 18, respectively;
d) a heavy chain variable (V_{H}) region comprising the CDR sequences SEQ ID NOs: 19, 20, 21 and a light chain variable (V_{L}) region comprising CDR sequences SEQ ID NOs: 22, 23, and 24, respectively;
e) the heavy chain variable (V_{H}) and light chain variable (V_{L}) regions comprising the CDR sequences SEQ ID NOS. 1-3 and 7-9, respectively;
f) the heavy chain variable (V_{H}) and light chain variable (V_{L}) regions comprising the CDR sequences SEQ ID NOS. 4-6 and 10-12, respectively;
g) the heavy chain variable (V_{H}) and light chain variable (V_{L}) regions comprising SEQ ID NO. 13 and SEQ ID NO. 14, respectively;
h) the heavy chain variable (V_{H}) and light chain variable (V_{L}) regions comprising the CDR sequences SEQ ID NOS. 15-17 and 21-23, respectively;
i) the heavy chain variable (V_{H}) and light chain variable (V_{L}) regions comprising the CDR sequences SEQ ID NOS. 18-20 and 24-26, respectively;
j) the heavy chain variable (V_{H}) and light chain variable (V_{L}) regions comprising SEQ ID NO. 27 and SEQ ID NO. 28, respectively;
k) the heavy chain variable (V_{H}) and light chain variable (V_{L}) regions comprising the CDR sequences SEQ ID NOS. 29-31 and 35-37, respectively;
l) the heavy chain variable (V_{H}) and light chain variable (V_{L}) regions comprising the CDR sequences SEQ ID NOS. 32-34 and 38-40, respectively;
m) the heavy chain variable (V_{H}) and light chain variable (V_{L}) regions comprising SEQ ID NO. 41 and SEQ ID NO. 42, respectively;
n) the heavy chain variable (V_{H}) and light chain variable (V_{L}) regions comprising the CDR sequences SEQ ID NOS. 43-45 and 49-51, respectively;
o) the heavy chain variable (V_{H}) and light chain variable (V_{L}) regions comprising the CDR sequences SEQ ID NOS. 46-48 and 52-54, respectively;
p) the heavy chain variable (V_{H}) and light chain variable (V_{L}) regions comprising SEQ ID NO. 55 and SEQ ID NO. 56, respectively;
or a derivative of any one of a)-p);
wherein the antibody or antigen binding fragment thereof specifically binds to human progranulin (PGRN).

35. The isolated antibody or antigen binding fragment thereof of claim 34, comprising:
a) a heavy chain variable (V_{H}) region comprising SEQ ID NO: 25 and a light chain variable (V_{L}) region comprising SEQ ID NO:27;
b) a heavy chain variable (V_{H}) region comprising SEQ ID NO: 29 and a light chain variable (V_{L}) region comprising SEQ ID NO:31;
c) a heavy chain variable (V_{H}) region comprising SEQ ID NO: 33 and a light chain variable (V_{L}) region comprising SEQ ID NO:35; or
d) a heavy chain variable (V_{H}) region comprising SEQ ID NO: 37 and a light chain variable (V_{L}) region comprising SEQ ID NO:39.

36. An antibody of claim 34 wherein the antibody is internalized into a cell that expresses hu PGRN in vitro and/or in vivo.

37. An antibody of claim 34 that competes with trastuzumab for binding to hu PGRN receptor on the cell.

38. The antibody of claim 34 selected from the group consisting of antibodies 10C8, 16C11, 14A6, and 10B3.

39. An antibody of claim 34 that does not compete with trastuzumab for binding to hu PGRN receptor on the cell.

40. The antibody of claim 39 selected from the group consisting of antibodies 10C8, 16C11, 14A6, and 10B3.

41. A combination of antibodies of claim 34, wherein at least one antibody competes with trastuzumab for binding to hu PGRN receptor on the cell and at least one antibody does not compete with trastuzumab for binding to hu PGRN receptor on the cell.

42. The antibody of claim 34 that is an isolated monoclonal antibody.

43. The antibody of claim 42 wherein the monoclonal antibody is a human monoclonal antibody.

44. The antibody of claim 34 wherein said antibody is derived from a human antibody, human IgG, human IgG1, human IgG2, human IgG2a, human IgG2b, human IgG3, human IgG4, human IgM, human IgA, human IgA1, human IgA2, human IgD, human IgE, canine antibody, canine IgGA, canine IgGB, canine IgGC, canine IgGD, chicken antibody, chicken IgA, chicken IgD, chicken IgE, chicken IgG, chicken IgM, chicken IgY, goat antibody, goat IgG, mouse antibody, mouse IgG, pig antibody, rat antibody, Ilaman antibody, alpacan antibody, shark antibody and a camel antibody.

45. A derivative of claim 34, optionally selected from the group consisting of an F_{ab}, F_{ab2}, Fab′ single chain antibody, Fᵥ, single chain, mono-specific antibody, bispecific antibody, trimeric antibody, multi-specific antibody, multivalent antibody, chimeric antibody, canine-human chimeric antibody, canine-mouse chimeric antibody, antibody comprising a canine Fc, humanized antibody, human antibody, caninized antibody, CDR-grafted antibody, shark antibody, and a nanobody.

46. A derivative of claim 34 comprising a detectable label fixably attached thereto, optionally wherein the detectable label is selected from the group consisting of fluorescein, DyLight, Cy3, Cy5, FITC, HiLyte Fluor 555, HiLyte Fluor 647, 5-carboxy-2,7-dichlorofluorescein, 5-carboxyfluorescein, 5-FAM, hydroxy tryptamine, 5-hydroxy tryptamine (5-HAT), 6-carboxyfluorescein (6-FAM), FITC, 6-carboxy-1,4-dichloro-2′,7′-dichlorofluorescein (TET), 6-carboxy-1,4-dichloro-2′,4′,5′,7′-tetrachlorofluorescein (HEX), 6-carboxy-4′,5′-dichloro-2′,7′-dimethoxyfluorescein (6-JOE), an Alexa fluor, Alexa fluor 350, Alexa fluor 405, Alexa fluor 430, Alexa fluor 488, Alexa fluor 500, Alexa fluor 514, Alexa fluor 532, Alexa fluor 546, Alexa fluor 555, Alexa fluor 568, Alexa fluor 594, Alexa fluor 610, Alexa fluor 633, Alexa fluor 635, Alexa fluor 647, Alexa fluor 660, Alexa fluor 680, Alexa fluor 700, Alexa fluor 750, a BODIPY fluorophores, BODIPY 492/515, BODIPY 493/503, BODIPY 500/510, BODIPY 505/515, BODIPY 530/550, BODIPY 542/563, BODIPY 558/568, BODIPY 564/570, BODIPY 576/589, BODIPY 581/591, BODIPY 630/650-X, BODIPY 650/665-X, BODIPY 665/676, FL, FL ATP, FI-Ceramide, R6G SE, TMR, TMR-X conjugate, TMR-X, SE, TR, TR ATP, TR-X SE, a rhodamine, rhodamine 110, rhodamine 123, rhodamine B, rhodamine B 200, rhodamine BB, rhodamine BG, rhodamine B extra, 5-carboxytetramethylrhodamine (5-TAMRA), 5 GLD, 6-carboxyrhodamine 6G, Lissamine, Lissamine Rhodamine B, Phallicidine, Phalloidine, rhodamine red, Rhod-2, 6-carboxy-X-rhodamine (ROX), carboxy-X-rhodamine (5-ROX), Sulphorhodamine B can C, Sulphorhodamine G Extra, 6-carboxytetramethyirhodamine (TAMRA), tetramethylrhodamine (TRITC), rhodamine WT, Texas Red, and Texas Red-X.

47. The antibody of claim 34 comprising an effector moiety attached thereto, optionally wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical.

48. The antibody of claim 47 further comprising a cleavable linker positioned between the antibody and the effector moiety, wherein said cleavable linker releases the effector moiety into or within a cell.

49. An isolated polynucleotide encoding antibody or derivative of claim 34, optionally wherein a nucleic acid sequence of at least one of SEQ ID NOS. 57-64.

50. An expression vector comprising one or more polynucleotides of claim 49.

51. A host cell comprising the isolated polynucleotide of claim 49.

52. A composition comprising at least one antibody or derivative of claim 34; and, a pharmaceutically acceptable carrier.

53. A composition comprising at least one isolated polynucleotide of claim 49; and, a pharmaceutically acceptable carrier.

54. A method for detecting hu PGRN on a cell, the method comprising contacting a test biological sample with an antibody or derivative of claim 34 and detecting the antibody bound to the biological sample or component thereof, optionally wherein:
the test biological sample comprises mammalian cells, tissue(s), and/or blood; and/or,
the method is an in vivo method or an in vitro method.

55. The method of claim 54 wherein the cell is selected from the group consisting of a breast cancer, adenocarcinoma epidermal growth factor receptor (EGF-R) breast cancer, basal breast carcinoma, estrogen receptor (ER) luminal breast cancer, ER positive tamoxifen resistant luminal breast cancer, letrozole resistant luminal breast cancer, biliary cancer, bladder cancer, brain cancer, glioblastoma, colorectal cancer, epidermoid carcinoma, squamous carcinoma, esophogeal cancer, gastric cancer, hepatocellular cancer, kidney/renal cancer, laryngeal cancer, lung cancer, non-small-cell lung carcinoma, lung adenocarcinoma, mesothelioma, myeloma/leukemia, ovarian cancer, prostate cancer, and uterine cancer cell.

56. The method of claim 54, further comprising comparing the amount of binding to the test biological sample or components thereof to the amount of binding to a control biological sample or components thereof, wherein increased binding to the test biological sample or components thereof relative to the control biological sample or components thereof indicates the presence of a cell expressing hu PGRN in the test biological sample.

57. A method for treating, preventing and/or ameliorating cancer in a mammal comprising administering to the mammal at least one effective dose of a pharmaceutical composition comprising an antibody or derivative of claim 34, optionally wherein:
the antibody or derivative is administered as a drug conjugate;
the antibody or derivative is administered to the mammal in a dosage amount of about 1 to 50 mg/kg; and/or,
multiple doses of the composition are administered to the mammal.

58. The method of claim 57 wherein the cancer is selected from the group consisting of breast cancer, adenocarcinoma epidermal growth factor receptor (EGF-R) breast cancer, basal breast carcinoma, estrogen receptor (ER) luminal breast cancer, ER positive tamoxifen resistant luminal breast cancer, letrozole resistant luminal breast cancer, biliary cancer, bladder cancer, brain cancer, glioblastoma, colorectal cancer, epidermoid carcinoma, squamous carcinoma, esophogeal cancer, gastric cancer, hepatocellular cancer, kidney/renal cancer, laryngeal cancer, lung cancer, non-small-cell lung carcinoma, lung adenocarcinoma, mesothelioma, myeloma/leukemia, ovarian cancer, prostate cancer, and uterine cancer.

59. The method of claim 57 wherein the drug conjugate comprises a cytotoxic effector moiety, optionally wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical.

60. The method of claim 59 wherein the drug conjugate comprises a cleavable linker positioned between the antibody or derivative and the effector moiety, wherein said cleavable linker releases the effector moiety into or within a cell.

61. A kit for detecting the expression of hu PGRN in or on a cell, the kit comprising an antibody or derivative of claim 34 and instructions for use, optionally wherein the antibody or derivative is in lyophilized form.

62. The kit of claim 61 wherein the cell is selected from the group consisting of a breast cancer, adenocarcinoma epidermal growth factor receptor (EGF-R) breast cancer, basal breast carcinoma, estrogen receptor (ER) luminal breast cancer, ER positive tamoxifen resistant luminal breast cancer, letrozole resistant luminal breast cancer, biliary cancer, bladder cancer, brain cancer, glioblastoma, colorectal cancer, epidermoid carcinoma, squamous carcinoma, esophogeal cancer, gastric cancer, hepatocellular cancer, kidney/renal cancer, laryngeal cancer, lung cancer, non-small-cell lung carcinoma, lung adenocarcinoma, mesothelioma, myeloma/leukemia, ovarian cancer, prostate cancer, and uterine cancer cell.


SEQUENCE LISTING (65 sequences)

SEQ ID NO: 1 - protein, 7 aa, Homo sapiens
GYTLTSY

SEQ ID NO: 2 - protein, 6 aa, Homo sapiens
SAYNGN

SEQ ID NO: 3 - protein, 19 aa, Homo sapiens
ELLWFGELLSQYYYYGMDV

SEQ ID NO: 4 - protein, 11 aa, Homo sapiens
RASQNISRWLA

SEQ ID NO: 5 - protein, 7 aa, Homo sapiens
RASSLET

SEQ ID NO: 6 - protein, 8 aa, Homo sapiens
QQYNSYYT

SEQ ID NO: 7 - protein, 5 aa, Homo sapiens
SYGIS

SEQ ID NO: 8 - protein, 17 aa, Homo sapiens
WISAYNGNTNYAQKLQG

SEQ ID NO: 9 - protein, 19 aa, Homo sapiens
ELLWFGELLSQYYYYGMDV

SEQ ID NO: 10 - protein, 11 aa, Homo sapiens
RASQNISRWLA

SEQ ID NO: 11 - protein, 7 aa, Homo sapiens
RASSLET

SEQ ID NO: 12 - protein, 8 aa, Homo sapiens
QQYNSYYT

SEQ ID NO: 13 - protein, 128 aa, Homo sapiens
QVQLVQSGAEVKKPGASVKVSCKASGYTLTSYGISWVRQAPGQGLEWMGWISAYNGNTNYAQKLQGRVTMTTDTSTSTAYMELRSLRSDDTAVYYCARELLWFGELLSQYYYYGMDVWGQGTTVTVSS

SEQ ID NO: 14 - protein, 106 aa, Homo sapiens
DIQMTQSPSTLSASVGDRVTITCRASQNISRWLAWYQQKPGKAPKLLIFRASSLETGVPSRFGGSGSGTEFTLTISSLQPDDFATYYCQQYNSYYTFGQGTRLEIK

SEQ ID NO: 15 - protein, 7 aa, Homo sapiens
GFTFSSY

SEQ ID NO: 16 - protein, 6 aa, Homo sapiens
WYDGSK

SEQ ID NO: 17 - protein, 8 aa, Homo sapiens
DRGIGGDY

SEQ ID NO: 18 - protein, 17 aa, Homo sapiens
KSSQTVLYSSNNKNYLA

SEQ ID NO: 19 - protein, 7 aa, Homo sapiens
WASTRES

SEQ ID NO: 20 - protein, 9 aa, Homo sapiens
QQYYSTPYT

SEQ ID NO: 21 - protein, 5 aa, Homo sapiens
SYAMH

SEQ ID NO: 22 - protein, 17 aa, Homo sapiens
VIWYDGSKKYNADSVKG

SEQ ID NO: 23 - protein, 8 aa, Homo sapiens
DRGIGGDY

SEQ ID NO: 24 - protein, 17 aa, Homo sapiens
KSSQTVLYSSNNKNYLA

SEQ ID NO: 25 - protein, 7 aa, Homo sapiens
WASTRES

SEQ ID NO: 26 - protein, 9 aa, Homo sapiens
QQYYSTPYT

SEQ ID NO: 27 - protein, 117 aa, Homo sapiens
QVQLVESGGGVVQPGRSLRLSCAASGFTFSSYAMHWVRQAPGKGLEWVAVIWYDGSKKYNADSVKGRFTISRDNSKNTLYLQMNSLRAEDTAVYYCARDRGIGGDYWGQGTLVTVSS

SEQ ID NO: 28 - protein, 113 aa, Homo sapiens
DIVMTQSPDSLAVSLGERATINCKSSQTVLYSSNNKNYLAWYQQKPGQPPKLLIYWASTRESGVPDRFSGGGSGTDFTLTISSLQAEDVAVYYCQQYYSTPYTFGQGTKLEIN

SEQ ID NO: 29 - protein, 7 aa, Homo sapiens
GFTFSNY

SEQ ID NO: 30 - protein, 6 aa, Homo sapiens
WYDGSN

SEQ ID NO: 31 - protein, 9 aa, Homo sapiens
GHSVSGGDY

SEQ ID NO: 32 - protein, 11 aa, Homo sapiens
RASQSVNSFLN

SEQ ID NO: 33 - protein, 7 aa, Homo sapiens
AASSLQS

SEQ ID NO: 34 - protein, 9 aa, Homo sapiens
QQSYSTPYT

SEQ ID NO: 35 - protein, 5 aa, Homo sapiens
NYGMH

SEQ ID NO: 36 - protein, 17 aa, Homo sapiens
VIWYDGSNKYYADSVKG

SEQ ID NO: 37 - protein, 9 aa, Homo sapiens
GHSVSGGDY

SEQ ID NO: 38 - protein, 11 aa, Homo sapiens
RASQSVNSFLN

SEQ ID NO: 39 - protein, 7 aa, Homo sapiens
AASSLQS

SEQ ID NO: 40 - protein, 9 aa, Homo sapiens
QQSYSTPYT

SEQ ID NO: 41 - protein, 118 aa, Homo sapiens
QVQLVESGGGVVQPGRSLRLSCAASGFTFSNYGMHWVRQAPGKGLEWVAVIWYDGSNKYYADSVKGRFTISRDNSKNTLYLQMNSLRAEDTAVYYCARGHSVSGGDYWGQGTLVTVSS

SEQ ID NO: 42 - protein, 107 aa, Homo sapiens
DIQMTQSPSSLSASVGDRVTITCRASQSVNSFLNWYQQTPGKAPKLLIYAASSLQSGVPPRFSGSGSGTDFTLTISSLQPEDFATYYCQQSYSTPYTFGQGTKLEIN

SEQ ID NO: 43 - protein, 7 aa, Homo sapiens
GFTLSGY

SEQ ID NO: 44 - protein, 6 aa, Homo sapiens
WYDGSD

SEQ ID NO: 45 - protein, 9 aa, Homo sapiens
DRGAVAGDY

SEQ ID NO: 46 - protein, 17 aa, Homo sapiens
KSSQSVLYNSNNKNLLA

SEQ ID NO: 47 - protein, 7 aa, Homo sapiens
WASTRES

SEQ ID NO: 48 - protein, 9 aa, Homo sapiens
QQYYGTPYT

SEQ ID NO: 49 - protein, 5 aa, Homo sapiens
GYGMH

SEQ ID NO: 50 - protein, 17 aa, Homo sapiens
VIWYDGSDKNYADSVKG

SEQ ID NO: 51 - protein, 9 aa, Homo sapiens
DRGAVAGDY

SEQ ID NO: 52 - protein, 17 aa, Homo sapiens
KSSQSVLYNSNNKNLLA

SEQ ID NO: 53 - protein, 7 aa, Homo sapiens
WASTRES

SEQ ID NO: 54 - protein, 9 aa, Homo sapiens
QQYYGTPYT

SEQ ID NO: 55 - protein, 118 aa, Homo sapiens
QVQLVESGGGVVQPGRSLRLSCAASGFTLSGYGMHWVRQAPDKGLEWVAVIWYDGSDKNYADSVKGRFTISRDNSKNTLYLQMNSLRAEDTAVYYCARDRGAVAGDYWGQGTLVTVSS

SEQ ID NO: 56 - protein, 113 aa, Homo sapiens
DIVMTQSPDSLAVSLGERATINCKSSQSVLYNSNNKNLLAWFRQKPGQPPKLLIYWASTRESGVPDRFSGSGSGTDFTLTISSLQAEDVAVYFCQQYYGTPYTFGQGTKLEIK

SEQ ID NO: 57 - DNA, 385 bp, Homo sapiens
  source 1..385: mol_type = genomic DNA
caggttcagctggtgcagtctggcgctgaggtgaagaagcctggggcctcagtgaaggtctcctgcaaggcttctggttacaccttaaccagctatggtatcagctgggtgcgacaggcccctggacaaggtcttgagtggatgggatggatcagcgcttacaatggtaacacaaattatgcacagaagctccagggcagagtcaccatgaccacagacacatccacgagcacagcctacatggagctgaggagcctgagatctgacgacacggccgtgtattattgtgcgagagagttattatggttcggggagttattatcccaatactattactacggtatggacgtctggggccaagggaccacggtcaccgtctcctcag

SEQ ID NO: 58 - DNA, 319 bp, Homo sapiens
  source 1..319: mol_type = genomic DNA
gacatccagatgacccagtctccttccaccctgtctgcatctgtaggagacagagtcaccatcacttgccgggccagtcaaaatattagtcgctggttggcctggtatcagcagaaaccagggaaagcccctaaactcctgatctttagggcatctagtttagaaactggggtcccatcaaggttcggcggcagtggatctgggacagaattcactctcaccatcagcagcctgcagcctgatgattttgcaacttattactgccaacagtataatagttattacacttttggccaggggaccaggctggagatcaaac

SEQ ID NO: 59 - DNA, 352 bp, Homo sapiens
  source 1..352: mol_type = genomic DNA
caggtgcagctggtggagtctgggggaggcgtggtccagcctgggaggtccctgagactctcctgtgcagcgtctggattcaccttcagtagttatgccatgcactgggtccgccaggctccaggcaaggggctggagtgggtggcagttatatggtatgatggaagtaaaaaatacaatgcagactccgtgaagggccgattcaccatctccagagacaattccaagaacacgctgtatctgcaaatgaacagcctgagagccgaggacacggctgtgtattactgtgcgagagatagggggataggtggggactactggggccagggaaccctggtcaccgtctcctcag

SEQ ID NO: 60 - DNA, 340 bp, Homo sapiens
  source 1..340: mol_type = genomic DNA
gacatcgtgatgacccagtctccagactccctggctgtgtctctgggcgagagggccaccatcaactgcaagtccagtcagactgttttatacagctccaacaataagaactacttagcttggtaccagcagaagccaggacagcctcctaagctgctcatttactgggcatctacccgggaatccggggtccctgaccgattcagtggcggcgggtctgggacagatttcactctcaccatcagcagcctgcaggctgaagatgtggcagtttattactgtcaacaatattatagtactccgtacacttttggccaggggaccaagctggagatcaacc

SEQ ID NO: 61 - DNA, 354 bp, Homo sapiens
  source 1..354: mol_type = genomic DNA
caggtgcagctggtggagtctgggggaggcgtggtccagcctgggaggtccctgagactctcctgtgcagcgtctggattcaccttcagtaactatggcatgcactgggtccgccaggctccaggcaaggggctggagtgggtggcagttatatggtatgatggaagtaataaatactatgcagactccgtgaagggccgcttcaccatctccagagacaattccaagaacacgctgtatctgcaaatgaacagcctgagagccgaggacacggctgtctattactgtgcgagagggcactctgtttcggggggtgactactggggccagggaaccctggtcaccgtctcctca

SEQ ID NO: 62 - DNA, 321 bp, Homo sapiens
  source 1..321: mol_type = genomic DNA
gacatccagatgacccagtctccatcctccctgtctgcatctgtaggagacagagtcaccatcacttgccgggcaagtcagagcgtgaacagcttcttgaattggtatcagcagacaccagggaaagcccctaaactcctgatctatgctgcatccagtttgcaaagtggggtcccaccaaggttcagtggcagtggatctgggacagatttcactctcaccatcagcagtctgcaacctgaagattttgcaacttactactgtcaacagagttacagtaccccgtacacttttggccaggggaccaagctggagatcaac

SEQ ID NO: 63 - DNA, 355 bp, Homo sapiens
  source 1..355: mol_type = genomic DNA
caggtgcagctggtggagtctgggggaggcgtggtccagcctgggaggtccctgagactctcctgtgcagcgtctggattcaccctcagtggctatggcatgcactgggtccgccaggctccagacaaggggctggagtgggtggcagttatatggtatgatggaagtgataaaaactatgcagactccgtgaagggccgattcaccatctccagagacaattccaagaacacgctgtatctgcaaatgaacagcctgagagccgaggacacggctgtgtattactgtgcgagagatcgaggagcagtggctggtgactactggggccagggaaccctggtcaccgtctcctcag

SEQ ID NO: 64 - DNA, 340 bp, Homo sapiens
  source 1..340: mol_type = genomic DNA
gacatcgtgatgacccagtctccagactccctggctgtgtctctgggcgagagggccaccatcaactgcaagtccagccagagtgttttatacaactccaacaataagaacctcttagcctggttccggcagaaaccgggacagcctcctaagttgctcatttactgggcgtctacccgggaatccggggtccctgaccggttcagtggcagcgggtctgggacagatttcactctcaccatcagcagcctgcaggctgaagatgtggcagtttatttctgtcagcaatattatggtactccgtacacttttggccaggggaccaagctggagatcaaac

SEQ ID NO: 65 - DNA, 21 bp, Homo sapiens
  source 1..21: mol_type = genomic DNA
ggctacaccctgaccagctac